SEQUENCE LISTING (83 sequences)

SEQ ID NO: 1 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 2 - protein, 1170 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 3 - protein, 1207 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 4 - protein, 116 aa, Homo sapiens
MDYYRKYAAIFLVTLSVFLHVLHSAPDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 5 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 6 - protein, 91 aa, Homo sapiens [sequence omitted]

SEQ ID NO: 7 - protein, 247 aa, Homo sapiens
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 8 - protein, 160 aa, Homo sapiens
MVPSAGQLALFALGIVLAACQALENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 9 - protein, 169 aa, Homo sapiens
MTAGRRMEMLCAGRVPALLLCLGFHLLQAVLSTTVIPSCIPGESSDNCTALVQTEDNPRVAQVSITKCSSDMNGYCLHGQCIYLVDMSQNYCRCEVGYTGVRCEHFFLTVHQPLSKEYVALTVILIILFLITVVGSTYYFCRWYRNRKSKEPKKEYERVTSGDPELPQV

SEQ ID NO: 10 - protein, 208 aa, Homo sapiens
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTTILAVVAVVLSSVCLLVIVGLLMFRYHRRGGYDVENEEKVKLGMTNSH

SEQ ID NO: 11 - protein, 252 aa, Homo sapiens
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQLRRQYVRKYEGEAEERKKLRQENGNVHAIA

SEQ ID NO: 12 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 13 - protein, 98 aa, Homo sapiens
MKVSAVLLCLLLMTAAFNPQGLAQPDALNVPSTCCFTFSSKKISLQRLKSYVITTSRCPQKAVIFRTKLGKEICADPKEKWVQNYMKHLGRKAHTLKT

SEQ ID NO: 14 - protein, 150 aa, Homo sapiens
MNLWLLACLVAGFLGAWAPAVHTQGVFEDCCLAYHYPIGWAVLRRAWTYRIQEVSGSCNLPAAIFYLPKRHRKVCGNPKSREVQRAMKLLDARNKVFAKLHHNTQTFQAGPHAVKKLSSGNSKLSSSKFSNPISSSKRNVSLLISANSGL

SEQ ID NO: 15 - protein, 114 aa, Homo sapiens
MSLLSSRAARVPGPSSSLCALLVLLLLLTQPGPIASAGPAAAVLRELRCVCLQTTQGVHPKMISNLQVFAIGPQCSKVEVVASLKNGKEICLDPEAPFLKKVIQKILDGGNKEN

SEQ ID NO: 16 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 17 - protein, 155 aa, Homo sapiens
MVLSGALCFRMKDSALKVLYLHNNQLLAGGLHAGKVIKGEEISVVPNRWLDASLSPVILGVQGGSQCLSCGVGQEPTLTLEPVNIMELYLGAKESKSFTFYRRDMGLTSSFESAAYPGWFLCTVPEADQPVRLTQLPENGGWNAPITDFYFQQCD

SEQ ID NO: 18 - protein, 170 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERRRPKGRGKRRREKQRPTDCHLCGDAVPRR

SEQ ID NO: 19 - protein, 469 aa, Homo sapiens
MHSFPPLLLLLFWGVVSHSFPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 20 - protein, 402 aa, Homo sapiens [sequence omitted]

SEQ ID NO: 21 - protein, 120 aa, Homo sapiens
MHPLLNPLLLALGLMALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRD

SEQ ID NO: 22 - protein, 152 aa, Homo sapiens
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 23 - protein, 99 aa, Homo sapiens
MKVSAALLCLLLMAATFSPQGLAQPDSVSIPITCCFNVINRKIPIQRLESYTRITNIQCPKEAVIFKTKRGKEVCADPKERWVRDSMKHLDQIFQNLKP

SEQ ID NO: 24 - protein, 317 aa, Homo sapiens [sequence omitted]

SEQ ID NO: 25 - protein, 97 aa, Homo sapiens
MKVSAALLWLLLIAAAFSPQGLAGPASVPTTCCFNLANRKIPLQRLESYRRITSGKCPQKAVIFKTKLAKDICADPKKKWVQDSMKYLDQKSPTPKP

SEQ ID NO: 26 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 27 - protein, 314 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 28 - protein, 583 aa, Homo sapiens
MFLSMVSKIRTFGWVQNPGKFENLKRVVQVFDRNSKVHNEVKNIKIPTLVKESKIQKELVAIMNQHDLIYTYKELVGTGTSIRSEAPCDAIIQATIADQGNKKGYIDNWSSDGFLRWAHALGFIEYINKSDSFVITDVGLAYSKSADGSAIEKEILIEAISSYPPAIRILTLLEDGQHLTKFDLGKNLGFSGESGFTSLPEGILLDTLANAMPKDKGEIRNNWEGSSDKYARMIGGWLDKLGLVKQGKKEFIIPTLGKPDNKEFISHAFKITGEGLKVLRRAKGSTKFTRVPKRVYWEMLATNLTDKEYVRTRRALILEILIKAGSLKIEQIQDNLKKLGFDEVIETIENDIKGLINTGIFIEIKGRFYQLKDHILQFVIPNRGVTKQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 29 - protein, 92 aa, Homo sapiens
MTKLEEHLEGIVNIFHQYSVRKGHFDTLSKGELKQLLTKELANTIKNIKDKAVIDEIFQGLDANQDEQVDFQEFISLVAIALKAAHYHTHKE

SEQ ID NO: 30 - protein, 92 aa, Homo sapiens; identical to SEQ ID NO: 29

SEQ ID NO: 31 - protein, 89 aa, Homo sapiens
MKGLAAALLVLVCTMALCSCAQVGTNKELCCLVYTSWQIPQKFIVDYSETSPQCPKPGVILLTKRGRQICADPNKKWVQKYISDLKLNA

SEQ ID NO: 32 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 33 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 34 - protein, 240 aa, Homo sapiens
MLALLCSCLLLAAGASDAWTGEDSAEPNSDSAEWIRDMYAKVTEIWQEVMQRRDDDGALHAACQVQPSATLDAAQPRVTGVVLFRQLAPRAKLDAFFALEGFPTEPNSSSRAIHVHQFGDLSQGCESTGPHYNPLAVPHPQHPGDFGNFAVRDGSLWRYRAGLAASLAGPHSIVGRAVVVHAGEDDLGRGGNQASVENGNAGRRLACCVVGVCGPGLWERQAREHSERKKRRRESECKAA

SEQ ID NO: 35 - protein, 120 aa, Homo sapiens
MKVSVAALSCLMLVTALGSQARVTKDAETEFMMSKLPLENPVLLDRFHATSADCCISYTPRSIPCSLLESYFETNSECSKPGVIFLTKKGRRFCANPSDKQVQVCMRMLKLDTRIKTRKN

SEQ ID NO: 36 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 37 - protein, 92 aa, Homo sapiens
MSELEKAMVALIDVFHQYSGREGDKHKLKKSELKELINNELSHFLEEIKEQEVVDKVMETLDNDGDGECDFQEFMAFVAMVTTACHEFFEHE

SEQ ID NO: 38 - protein, 174 aa, Homo sapiens
MSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKH

SEQ ID NO: 39 - protein, 217 aa, Homo sapiens
MATGSRTSLLLAFGLLCLPWLQEGSAFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 40 - protein, 381 aa, Homo sapiens
MPFSNSHNALKLRFPAEDEFPDLSAHNNHMAKVLTPELYAELRAKSTPSGFTLDDVIQTGVDNPGHPYIMTVGCVAGDEESYEVFKDLFDPIIEDRHGGYKPSDEHKTDLNPDNLQGGDDLDPNYVLSSRVRTGRSIRGFCLPPHCSRGERRAIEKLAVEALSSLDGDLAGRYYALKSMTEAEQQQLIDDHFLFDKPVSPLLLASGMARDWPDARGIWHNDNKTFLVWVNEEDHLRVISMQKGGNMKEVFTRFCTGLTQIETLFKSKDYEFMWNPHLGYILTCPSNLGTGLRAGVHIKLPNLGKHEKFSEVLKRLRLQKRGTGGVDTAAVGGVFDVSNADRLGFSEVELVQMVVDGVKLLIEMEQRLEQGQAIDDLMPAQK

SEQ ID NO: 41 - protein, 540 aa, Homo sapiens
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLT

SEQ ID NO: 42 - protein, 92 aa, Homo sapiens
MKLCVTVLSLLMLVAAFCSPALSAPMGSDPPTACCFSYTARKLPRNFVVDYYETSSLCSQPAVVFQTKRSKQVCADPSESWVQEYVYDLELN

SEQ ID NO: 43 - protein, 207 aa, Homo sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 44 - protein, 745 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 45 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 46 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 47 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 48 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 49 - protein, 406 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 50 - protein, 440 aa, Homo sapiens [sequence omitted]

SEQ ID NO: 51 - protein, 177 aa, Homo sapiens
MRERPRLGEDSSLISLFLQVVAFLAMVMGTHTYSHWPSCCPSKGQDTSEELLRWSTVPVPPLEPARPNRHPESCRASEDGPLNSRAISPWRYELDRDLNRLPQDLYHARCLCPHCVSLQTGSHMDPRGNSELLYHNQTVFYRRPCHGEKGTHKGYCLERRLYRVSLACVCVRPRVMG

SEQ ID NO: 52 - protein, 267 aa, Homo sapiens
MRLTVLCAVCLLPGSLALPLPQEAGGMSELQWEQAQDYLKRFYLYDSETKNANSLEAKLKEMQKFFGLPITGMLNSRVIEIMQKPRCGVPDVAEYSLFPNSPKWTSKVVTYRIVSYTRDLPHITVDRLVSKALNMWGKEIPLHFRKVVWGTADIMIGFARGAHGDSYPFDGPGNTLAHAFAPGTGLGGDAHFDEDERWTDGSSLGINFLYAATHELGHSLGMGHSSDPNAVMYPTYGNGDPQNFKLSQDDIKGIQKLYGKRSNSRKK

SEQ ID NO: 53 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 54 - protein, 1332 aa, Homo sapiens
MESHSRAGKSRKSAKFRSISRSLMLCNAKTSDDGSSPDEKYPDPFEISLAQGKEGIFHSSVQLADTSEAGPSSVPDLALASEAAQLQAAGNDRGKTCRRIFFMKESSTASSREKPGKLEAQSSNFLFPKACHQRARSNSTSVNPYCTREIDFPMTKKSAAPTDRQPYSLCSNRKSLSQQLDCPAGKAAGTSRPTRSLSTAQLVQPSGGLQASVISNIVLMKGQAKGLGFSIVGGKDSIYGPIGIYVKTIFAGGAAAADGRLQEGDEILELNGESMAGLTHQDALQKFKQAKKGLLTLTVRTRLTAPPSLCSHLSPPLCRSLSSSTCITKDSSSFALESPSAPISTAKPNYRIMVEVSLQKEAGVGLGIGLCSVPYFQCISGIFVHTLSPGSVAHLDGRLRCGDEIVEISDSPVHCLTLNEVYTILSHCDPGPVPIIVSRHPDPQVSEQQLKEAVAQAVENTKFGKERHQWSLEGVKRLESSWHGRPTLEKEREKNSAPPHRRAQKVMIRSSSDSSYMSGSPGGSPGSGSAEKPSSDVDISTHSPSLPLAREPVVLSIASSRLPQESPPLPESRDSHPPLRLKKSFEILVRKPMSSKPKPPPRKYFKSDSDPQKSLEERENSSCSSGHTPPTCGQEARELLPLLLPQEDTAGRSPSASAGCPGPGIGPQTKSSTEGEPGWRRASPVTQTSPIKHPLLKRQARMDYSFDTTAEDPWVRISDCIKNLFSPIMSENHGHMPLQPNASLNEEEGTQGHPDGTPPKLDTANGTPKVYKSADSSTVKKGPPVAPKPAWFRQSLKGLRNRASDPRGLPDPALSTQPAPASREHLGSHIRASSSSSSIRQRISSFETFGSSQLPDKGAQRLSLQPSSGEAAKPLGKHEEGRFSGLLGRGAAPTLVPQQPEQVLSSGSPAASEARDPGVSESPPPGRQPNQKTLPPGPDPLLRLLSTQAEESQGPVLKMPSQRARSFPLTRSQSCETKLLDEKTSKLYSISSQVSSAVMKSLLCLPSSISCAQTPCIPKEGASPTSSSNEDSAANGSAETSALDTGFSLNLSELREYTEGLTEAKEDDDGDHSSLQSGQSVISLLSSEELKKLIEEVKVLDEATLKQLDGIHVTILHKEEGAGLGFSLAGGADLENKVITVHRVFPNGLASQEGTIQKGNEVLSINGKSLKGTTHHDALAILRQAREPRQAVIVTRKLTPEAMPDLNSSTDSAASASAASDVSVESTAEATVCTVTLEKMSAGLGFSLEGGKGSLHGDKPLTINRIFKGAASEQSETVQPGDEILQLGGTAMQGLTRFEAWNIIKALPDGPVTIVIRRKSLQSKETTAAGDS

SEQ ID NO: 55 - protein, 109 aa, Homo sapiens
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 56 - protein, 110 aa, Homo sapiens; identical to SEQ ID NO: 45

SEQ ID NO: 57 - protein, 2813 aa, Homo sapiens [sequence omitted]

SEQ ID NO: 58 - protein, 277 aa, Homo sapiens; identical to SEQ ID NO: 1

SEQ ID NO: 59 - protein, 273 aa, Homo sapiens
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAASSLRNDSSSSNRKAKNPPGDSSLHWAAMALPALFSLIIGFAFGALYWKKRQPSLTRAVENIQINEEDNEISMLQEKEREFQEV

SEQ ID NO: 60 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 61 - protein, 345 aa, Homo sapiens
MISPVLILFSSFLCHVAIAGRTCPKPDDLPFSTVVPLKTFYEPGEEITYSCKPGYVSRGGMRKFICPLTGLWPINTLKCTPRVCPFAGILENGAVRYTTFEYPNTISFSCNTGFYLNGADSAKCTEEGKWSPELPVCAPIICPPPSIPTFATLRVYKPSAGNNSLYRDTAVFECLPQHAMFGNDTITCTTHGNWTKLPECREVKCPFPSRPDNGFVNYPAKPTLYYKDKATFGCHDGYSLDGPEEIECTKLGNWSAMPSCKASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 62 - protein, 120 aa, Homo sapiens
MKVSEAALSLLVLILIITSASRSQPKVPEWVNTPSTCCLKYYEKVLPRRLVVGYRKALNCHLPAIIFVTKRNREVCTNPNDDWVQEYIKDPNLPLLPTRNLSTVKIITAKNGQPQLLNSQ

SEQ ID NO: 63 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 64 - protein, 404 aa, Homo sapiens
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 65 - protein, 575 aa, Homo sapiens
MLGVLVLGALALAGLGFPAPAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDCDSGKVDGGDSGSGEPPPSPTPGSTLTPPAVGLVHSGLLIGISIASLCLVVALLALLCHLRKKQGAARAKMEYKCAAPSKEVVLQHVRTERTPQRL

SEQ ID NO: 66 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 67 - protein, 100 aa, Homo sapiens
MKLLAATVLLLTICSLEGALVRRQAKEPCVESLVSQYFQTVTDYGKDLMEKVKSPELQAEAKSYFEKSKEQLTPLIKKAGTELVNFLSYFVELGTQPATQ

SEQ ID NO: 68 - protein, 1474 aa, Homo sapiens
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 69 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCHFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 70 - protein, 267 aa, Homo sapiens
MPRSCCSRSGALLLALLLQASMEVRGWCLESSQCQDLTTESNLLECIRACKPDLSAETPMFPGNGDEQPLTENPRKYVMGHFRWDRFGRRNSSSSGSSGAGQKREDVSAGEDCGPLPEGGPEPRSDGAKPGPREGKRSYSMEHFRWGKPVGKKRRPVKVYPNGAEDESAEAFPLEFKRELTGQRLREGDGPDGPADDGAGAQADLEHSLLVAAEKKDEGPYRMEHFRWGSPPKDKRYGGFMTSEKSQTPLVTLFKNAIIKNAYKKGE

SEQ ID NO: 71 - protein, 412 aa, Homo sapiens
MAPPSVFAEVPQAQPVLVFKLTADFREDPDPRKVNLGVGAYRTDDCHPWVLPVVKKVEQKIANDNSLNHEYLPILGLAEFRSCASRLALGDDSPALKEKRVGGVQSLGGTGALRIGADFLARWYNGTNNKNTPVYVSSPTWENHNAVFSAAGFKDIRSYRYWDAEKRGLDLQGFLNDLENAPEFSIVVLHACAHNPTGIDPTPEQWKQIASVMKHRFLFPFFDSAYQGFASGNLERDAWAIRYFVSEGFEFFCAQSFSKNFGLYNERVGNLTVVGKEPESILQVLSQMEKIVRITWSNPPAQGARIVASTLSNPELFEEWTGNVKTMADRILTMRSELRARLEALKTPGTWNHITDQIGMFSFTGLNPKQVEYLVNEKHIYLLPSGRINVSGLTTKNLDYVATSIHEAVTKI

SEQ ID NO: 72 - protein, 199 aa, Homo sapiens
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 73 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 74 - protein, 1304 aa, Homo sapiens
MQLKIMPKKKRLSAGRVPLILFLCQMISALEVPLDPKLLEDLVQPPTITQQSPKDYIIDPRENIVIQCEAKGKPPPSFSWTRNGTHFDIDKDPLVTMKPGTGTLIINIMSEGKAETYEGVYQCTARNERGAAVSNNIVVRPSRSPLWTKEKLEPITLQSGQSLVLPCRPPIGLPPPIIFWMDNSFQRLPQSERVSQGLNGDLYFSNVLPEDTREDYICYARFNHTQTIQQKQPISVKVISVDELNDTIAANLSDTEFYGAKSSRERPPTFLTPEGNASNKEELRGNVLSLECIAEGLPTPIIYWAKEDGMLPKNRTVYKNFEKTLQIIHVSEADSGNYQCIAKNALGAIHHTISVRVKAAPYWITAPQNLVLSPGEDGTLICRANGNPKPRISWLTNGVPIEIAPDDPSRKIDGDTIIFSNVQERSSAVYQCNASNEYGYLLANAFVNVLAEPPRILTPANTLYQVIANRPALLDCAFFGSPLPTIEWFKGAKGSALHEDIYVLHENGTLEIPVAQKDSTGTYTCVARNKLGMAKNEVHLEIKDPTWIVKQPEYAVVQRGSMVSFECKVKHDHTLSLTVLWLKDNRELPSDERFTVDKDHLVVADVSDDDSGTYTCVANTTLDSVSASAVLSVVAPTPTPAPVYDVPNPPFDLELTDQLDKSVQLSWTPGDDNNSPITKFIIEYEDAMHKPGLWHHQTEVSGTQTTAQLKLSPYVNYSFRVMAVNSIGKSLPSEASEQYLTKASEPDKNPTAVEGLGSEPDNLVITWKPLNGFESNGPGLQYKVSWRQKDGDDEWTSVVVANVSKYIVSGTPTFVPYLIKVQALNDMGFAPEPAVVMGHSGEDLPMVAPGNVRVNVVNSTLAEVHWDPVPLKSIRGHLQGYRIYYWKTQSSSKRNRRHIEKKILTFQGSKTHGMLPGLEPFSHYTLNVRVVNGKGEGPASPDRVFNTPEGVPSAPSSLKIVNPTLDSLTLEWDPPSHPNGILTEYTLKYQPINSTHELGPLVDLKIPANKTRWTLKNLNFSTRYKFYFYAQTSAGSGSQITEEAVTTVDEAGILPPDVGAGKVQAVNPRISNLTAAAAETYANISWEYEGPEHVNFYVEYGVAGSKEEWRKEIVNGSRSFFGLKGLMPGTAYKVRVGAVGDSGFVSSEDVFETGPAMASRQVDIATQGWFIGLMCAVALLILILLIVCFIRRNKGGKYPVKEKEDAHADPEIQPMKEDDGTFGEYSDAEDHKPLKKGSRTPSDRTVKKEDSDDSLVDYGEGVNGQFNEDGSFIGQYSGKKEKEPAEGNESSEAPSPVNAMNSFV

SEQ ID NO: 75 - protein, 68 aa, Homo sapiens
ELDVCVEEAEGEAPWTWTGLCIFAALFLLSVSYSAALTLLMVQRFLSATRQGRPQTSLDYTNVLQPHA

SEQ ID NO: 76 - protein, 485 aa, Homo sapiens
MRKRAPQSEMAPAGVSLRATILCLLAWAGLAAGDRVYIHPFHLVIHNESTCEQLAKANAGKPKDPTFIPAPIQAKTSPVDEKALQDQLVLVAAKLDTEDKLRAAMVGMLANFLGFRIYGMHSELWGVVHGATVLSPTAVFGTLASLYLGALDHTADRLQAILGVPWKDKNCTSRLDAHKVLSALQAVQGLLVAQGRADSQAQLLLSTVVGVFTAPGLHLKQPFVQGLALYTPVVLPRSLDFTELDVAAEKIDRFMQAVTGWKTGCSLMGASVDSTLAFNTYVHFQGKMKGFSLLAEPQEFWVDNSTSVSVPMLSGMGTFQHWSDIQDNFSVTQVPFTESACLLLIQPHYASDLDKVEGLTFQQNSLNWMKKLSPRTIHLTMPQLVLQGSYDLQDLLAQAELPAILHTELNLQKLSNDRIRVGEVLNSIFFELEADEREPTESTQQLNKPEVLEVTLNRPFLFAVYDQSATALHFLGRVANPLSTA

SEQ ID NO: 77 - protein, 133 aa, Homo sapiens [sequence omitted]

SEQ ID NO: 78 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 79 - protein, 154 aa, Homo sapiens
MGLSDGEWQLVLNVWGKVEADIPGHGQEVLIRLFKGHPETLEKFDKFKHLKSEDEMKASEDLKKHGATVLTALGGILKKKGHHEAEIKPLAQSHATKHKIPVKYLEFISECIIQVLQSKHPGDFGADAQGAMNKALELFRKDMASNYKELGFQG

SEQ ID NO: 80 - protein, 496 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 81 - protein, 477 aa, Homo sapiens
MKSLPILLLLCVAVCSAYPLDGAARGEDTSMNLVQKYLENYYDLKKDVKQFVRRKDSGPVVKKIREMQKFLGLEVTGKLDSDTLEVMRKPRCGVPDVGHFRTFPGIPKWRKTHLTYRIVNYTPDLPKDAVDSAVEKALKVWEEVTPLTFSRLYEGEADIMISFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGPPPDSPETPLVPTEPVPPEPGTPANCDPALSFDAVSTLRGEILIFKDRHFWRKSLRKLEPELHLISSFWPSLPSGVDAAYEVTSKDLVFIFKGNQFWAIRGNEVRAGYPRGIHTLGFPPTVRKIDAAISDKEKNKTYFFVEDKYWRFDEKRNSMEPGFPKQIAEDFPGIDSKIDAVFEEFGFFYFFTGSSQLEFDPNAKKVTHTLKSNSWLNC

SEQ ID NO: 82 - protein, 120 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDS

SEQ ID NO: 83 - protein, 222 aa, Homo sapiens
MAEKPKLHYFNARGRMESTRWLLAAAGVEFEEKFIKSAEDLDKLRNDGYLMFQQVPMVEIDGMKLVQTRAILNYIASKYNLYGKDIKERALIDMYIEGIADLGEMILLLPVCPPEEKDAKLALIKEKIKNRYFPAFEKVLKSHGQDYLVGNKLSRADIHLVELLYYVEELDSSLISSFPLLKALKTRISNLPTVKKFLQPGSPRKPPMDEKSLEEARKIFRF

(19) United States
(12) Patent Application Publication
(10) Pub. No.: US 2014/0011879 A1
(43) Pub. Date: Jan. 9, 2014

(54) SERUM MARKERS FOR IDENTIFICATION OF CUTANEOUS SYSTEMIC SCLEROSIS SUBJECTS

(71) Applicants: Frederic Baribaud, Spring House, PA (US); Bidisha Dasgupta, Spring House, PA (US)

(72) Inventors: Frederic Baribaud, Spring House, PA (US); Bidisha Dasgupta, Spring House, PA (US)

(73) Assignee: JANSSEN BIOTECH, INC., Horsham, PA (US)

(21) Appl. No.: 13/825,060

(22) PCT Filed: Sep. 27, 2011
(86) PCT No.: PCT/US11/53449

§ 371 Date: Sep. 23, 2013

Related U.S. Application Data
(60) Provisional application No. 61/387,580, filed on Sep. 29, 2010.

(51) Int. Cl. G01N 33/68; G06F 19/00

(52) U.S. Cl. CPC G01N 33/6893; G06F 19/345
USPC 514/562; 435/7.92; 435/287.2; 702/20

(57) ABSTRACT

Tools for diagnosis and management of patients suspected of having or having been previously diagnosed with systemic sclerosis are based on the determination one or more of the markers described herein, specifically, the markers having the amino acid sequence of SEQ ID NOS: 1-62 and 66-76 in a sample from the subject. Specific marker ratios and subsets of markers and ratios identify a patient and further subclassify the patient. The information may be used prospectively to study the response of subclassified patients to existing or novel therapeutic strategies.

### BACKGROUND OF THE INVENTION

1. Field of the Invention

The present invention relates to methods and procedures for the use of serum biomarkers to predict clinical heterogeneity and response to biologic therapeutics in patients diagnosed with Systemic Sclerosis (SSc).

2. Description of the Related Art

Diffuse systemic sclerosis (SSc) is an autoimmune disease of unknown etiology that targets multiple organs including the skin, lungs, heart, gut, kidneys, muscles and joints. Diffuse SSc has a prevalence in the U.S. of 240 to 300 cases per million population with 20 new cases per million diagnosed each year (Mayes et al, 2003 Arthritis Rheum. 48(8):2246-55). The clinical course of diffuse SSc varies considerably. Early skin involvement typically progresses in a rapid fashion, and may be followed by stabilization and spontaneous improvement throughout the course of the disease. However, visceral involvement generally follows a progressive course, although stabilization of the disease may occur (Furst et al, 2007 Rheumatol. 34(5):1194-200).

At present, SSc patients are classified according to the degree of skin involvement (also known as “modified Rodnan skin score” or MRSS) and the presence of autoantibodies in the serum that have been shown to correlate with defined clinical phenotypes (Scl-70 or ANA titers). Patients are categorized as “diffuse,” or “limited” SSc based on extent of skin and internal organ involvement. Diffuse patients are further categorized as “early progressive diffuse” or “late improving” based on worsening or improvement of the MRSS over a 3-6 month period. To date, no serum markers have been identified that can characterize these subpopulations or the heterogeneity seen in SSc patient populations.

The effectiveness of treatment and clinical study design is impacted by the present inability to classify SSc subpopulations for randomization across treatment arms of a clinical trial. In addition, no markers exist to predict the SSc patients who will respond to treatment. Surrogate markers or biomarkers may be useful in answering these questions

Biomarkers are defined as “a characteristic that is objectively measured and evaluated as an indicator of normal biologic processes, pathogenic processes, or pharmacologic responses to a therapeutic intervention” (Biomarker Working Group, 2001. Clin. Pharm. and Therap. 69: 89-95). The definition of a biomarker has recently been further defined as proteins in which the change of expression may correlate with an increased risk of disease or progression, or which may be predictive of a response to a given treatment.

Although no clear biomarkers have been reported for SSc, several studies have shown that serum levels of certain cytokines and chemokines are either upregulated or downregulated in patients with SSc. Increased levels of IL-13 and IL-13-associated downstream mediators of inflammation and fibrosis (e.g., chemokine (C-C motif) ligand 2 (CCL-2) and TGF-β), have been widely reported to be elevated in the blood and affected tissues of diffuse SSc subjects (Hasegawa 1997 J. Rheumatol. 24(2):328-32; Mayes et al 2003 supra). A recent study demonstrated that SSc patients have higher circulating levels of Th-2 cytokines, such as CXCL-10 and CCL2. Other studies have reported elevated levels of IL-23 (Komura et al, 2008), endothelin (Silver et al, 2008 Rheumatology 47 Suppl 5:v25-6), and tissue inhibitor of metalloproteinase-1 (TIMP-1) (Yazawa et al, 2000 J Am Acad Dermatol. 42(1 Pt 1):70-5) in serum from SSc subjects.

Apart from these reports, a comprehensive interrogation of other serum cytokines and chemokines has not been conducted in diffuse SSc. Therefore, a unique set of markers that can classify the SSc population and are predictive of response (or non-response) to therapy has not yet been discovered.

Therefore, while a number of serum protein and non-protein markers of inflammation and systemic disease have been demonstrated to be modified during anti-TNFα treatment, a unique set of markers and a predictive algorithm have not, thus far, been discovered which is predictive of response or non-response for either all inflammatory diseases so treated or for specific diseases. Thus, a need exists for SSc makers for identification and classification of the disease.

### SUMMARY OF THE INVENTION

The invention comprises the use of multiple biomarkers to classify a subject suspected of having systemic sclerosis (SSc) as having SSc and, further, subclassifying the subject as having limited SSc or diffuse SSc or alternatively subclassifying the subject as belonging to a subset of diffuse SSc patients. In one embodiment, the concentration of markers in serum from a patent suspected of having SSc is elevated compared to a values from normal control subjects. In a specific embodiment, the concentration of two or more of the markers as compared to the concentration in a standard representing a normal control is at least two-fold higher.

In another embodiment, the concentrations of IL-17 and GST in the serum of a patient diagnosed with SSc are lower than in a standard representing patients diagnosed with limited SSc and the concentrations IL-13 and IgE are higher than in a standard representing patients diagnosed with limited SSc, indicating the patient has diffuse SSc. In another embodiment, in patients diagnosed with diffuse SSc, the concentrations of markers in the serum further classify the diffuse patients as early progressive diffuse (EP) or late improving diffuse (LI).

In another embodiment, specific marker sets identified in datasets from patients diagnosed with and previously classified as having diffuse or limited SSc, are used to monitor the clinical response of SSc patients to therapy.

The invention also provides a computer-based system for diagnosing a SSc in a subject, wherein the computer uses values from a patient's dataset to compare to a diagnostic index or an algorithm, such as a decision tree, wherein the dataset includes the serum concentrations of one or more markers described herein. In one embodiment, the computer-based system is a trained neural network for processing a patient dataset and produces an output wherein the dataset includes one or more serum marker concentrations described herein.

The invention further provides a device capable of processing and detecting serum markers in a specimen or sample obtained from subject suspected of having SSc. In one embodiment, the device compares the information produced by detection of one or more of the markers described herein into an algorithm for diagnosing and classifying a subject with SSc.

The invention also provides a kit comprising a device capable of processing and/or detecting serum markers in a specimen or sample obtained from an SSc patient wherein the serum marker concentrations are processed and/or detected, whereby the processed and/or detected serum marker level may used to calculate and index or used in an algorithm for diagnosing and subclassifying a subject suspected of having SSc.

### DETAILED DESCRIPTION OF THE INVENTION

### Abbreviations

CART, classification and regression tree model; CRP, C-reactive protein; EIA, Enzyme Immunoassay; ELISA, Enzyme Linked Immunoassay; FDR, false discovery rate; FPR, false positive rate; G-CSF, granulocyte colony stimulating factor; MAP, multi-analyte profile; SELDI, Surface Enhanced Laser Desorption and Ionization; IL, Interleukin; SSc, systemic sclerosis

### DEFINITIONS

A “biomarker” is defined as ‘a characteristic that is objectively measured and evaluated as an objective indicator of normal biological processes, pathogenic processes, or pharmacologic responses to a therapeutic intervention’ by the Biomarkers Definitions Working Group (Atkinson et al. 2001 Clin Pharm Therap 69(3):89-95). Thus, an anatomic or physiologic process can serve as a biomarker, for example, range of motion, as can levels of proteins, gene expression (mRNA), small molecules, metabolites or minerals, provided there is a validated link between the biomarker and a relevant physiologic, toxicologic, pharmacologic, or clinical outcome.

By “BDNF” is meant “brain-derived neurotrophic factor” also known as abrineurin, obsessive-compulsive disorder 1, OCD1 having an amino acid sequence as given in the SwissProt record, P 23560.

By “CCL2” is meant a C-C motif chemokine 2, GDCF-2, HC11, HSMCR30, MCAF, MCP1, MCP-1, MGC9434, Monocyte chemoattractant protein 1, Monocyte chemotactic and activating factor, monocyte chemotactic protein 1, monocyte secretory protein JE, SCYA2, small-inducible cytokine A2, SMC-CF having an amino acid sequence as given in the SwissProt record, P13500. CCL2 was discovered to function in the recruitment of monocytes to sites of injury and infection.

By “CCL5” is meant a C-C motif chemokine 5, D17S136E, EoCP, Eosinophil-chemotactic cytokine, MGC17164, RANTES, SCYA5, SISd, SIS-delta, Small-inducible cytokine A5, T cell-specific protein P228, T-cell-specific protein RANTES, TCP228 having an amino acid sequence as given in the SwissProt record, P13501.

By “CCL11” is meant “C-C motif chemokine 11,” also known as Eosinophil chemotactic protein, eotaxin, Eotaxin, MGC22554, SCYA11, Small-inducible cytokine All having an amino acid sequence as given in the SwissProt record, P51671.

By “CXCL5” is meant a C-X-C motif chemokine 5 also known as ENA78, ENA-78, ENA-78(1-78), Epithelial-derived neutrophil-activating protein 78, Neutrophil-activating peptide ENA-78, SCYB5, Small-inducible cytokine B5 having an amino acid sequence as given in the SwissProt record, P42830.

“CRP” or “C-Reactive Protein” is an acute phase reactant, which can be used as a general screening aid for inflammatory diseases, infections, and neoplastic diseases. In addition to its usual value as an acute phase reactant, CRP in large concentration (>5 mg/dL) predicts progression of erosions in rheumatoid arthritis. Elevated serum CRP is characteristic of bacterial, but not viral, meningitis or meningoencephalitis. Elevated concentrations of CRP are associated with risk of myocardial infarction in patients with stable and unstable angina and predict risk of first myocardial infarction and ischemic stroke in apparently healthy individuals. The Swiss-Prot Accession Number for CRP is P02741.

By “EGF” is meant “epidermal growth factor” which has also been known as urogastrone (URG) and HOMG4, Pro-epidermal growth factor having an amino acid sequence as given in the SwissProt record, P01133.

“Fibrinogen” is a proprotein which is cleaved by thrombin to form fibrin is the final common reaction of the coagulation cascade. Low levels of fibrinogen are seen in association with fibrinolysis and liver disease. A high level of fibrinogen is a risk factor for thrombosis and is a strong predictor of cardiovascular risk and stroke, particularly in young adults. Low-dose heparin and ACE-inhibitors reduce fibrinogen and risk of adverse cardiovascular events. The composition of fibrinogen is given by Swiss-Prot Accession Records Alpha chain P02671; Beta chain P02675; Gamma chain P02679.

By “GST” is meant “Glutathione S-Transferase alpha” having an amino acid sequence given in Swiss-Prot Accession Record P0826, and represents enzymes that utilize glutathione in reactions contributing to the transformation of a wide range of compounds, including carcinogens, therapeutic drugs, and products of oxidative stress.

By “IL13” is meant “interleukin 13” and is also known as ALRH, BHR1, MGC116786, MGC116788, MGC116789, NC30, P600 having an amino acid sequence as given in the SwissProt record, P35225.

By “IL17” is meant “interleukin 17” also known as CTLA8, CTLA-8, Cytotoxic T-lymphocyte-associated antigen 8, IL-17A, Interleukin-17A and having an amino acid sequence given by the NCBI accession record NP_{—}002181.

By “MPO” is meant “myeloperoxidase,” an enzyme capable of catalyzing the production of hypohalous acids, primarily hypochlorous acid in physiologic situations, and other toxic intermediates that greatly enhance PMN microbicidal activity and having an amino acid sequence as given in the SwissProt record, P051664.

By “IgE” is meant molecules comprising the immunoglobulin heavy constant epsilon sequence, exemplified by the amino acid sequence giving in SwissProt P01854, and encompasses IgE molecules of varying binding specificity encompassed by the definition and sequences defining the IgE class of human immunoglobulins.

By “VEGF” is meant vascular endothelial growth factor also known as MGC70609, MVCD1, vascular endothelial growth factor A, vascular permeability factor, VEGF-A, VPF and having an amino acid sequence as given in the SwissProt record, P15692.

By “serum level” of a marker is meant the concentration of the marker measured by one or more methods, such as an immunoassay, typically ex vivo on a sample prepared from a specimen such as blood. The immunoassay uses immunospecific reagents, typically antibodies, for each marker and the assay may be performed in a variety of formats including enzyme-coupled reactions, e.g., EIA, ELISA, RIA, or other direct or indirect probe. Other methods of quantifying the marker in the sample such electrochemical, fluorescence probe-linked detection are also possible. The assay may also be “multiplexed” wherein multiple markers are detected and quantitated during a single sample interrogation. The serum level can be measured by measuring all or a portion of the relevant protein marker as described herein. Any portion of the protein that allows identification of the presence of the protein is suitable for purposes of the methods of the present invention.

Predictive values help interpret the results of tests in the clinical setting. The diagnostic value of a procedure is defined by its sensitivity, specificity, predictive value and efficiency. Any test method will produce True Positive (TP), False Negative (FN), False Positive (FP), and True Negative (TN). The “sensitivity” of a test is the percentage of all patients with disease present or that do respond who have a positive test or (TP/TP+FN)×100%. The “specificity” of a test is the percentage of all patients without disease or who do not respond, who have a negative test or (TN/FP+TN)×100%. The likelihood ratio (LR) combines information contained in the sensitivity and specificity to provide information about how the odds of having a disease change given a positive or negative test result. The higher the likelihood ratio, the better the test can support the diagnosis. Mathematically, the likelihood ratios can be expressed as: Positive LR=sensitivity/1−specificity. The “predictive value” or “PV” of a test is a measure (%) of the times that the value (positive or negative) is the true value, i.e., the percent of all positive tests that are true positives is the Positive Predictive Value (PV+) or (TP/TP+FP)×100%. The “negative predictive value” (PV−) is the percentage of patients with a negative test who will not respond or (TN/FN+TN)×100%. The “accuracy” or “efficiency” of a test is the percentage of the times that the test gives the correct answer compared to the total number of tests or (TP+TN/TP+TN+FP+FN)×100%. The “error rate” calculates from those patients predicted to respond who did not and those patients who responded that were not predicted to respond or (FP+FN/TP+TN+FP+FN)×100%. The PV changes with a physician's clinical assessment of the presence or absence of disease or presence or absence of clinical response in a given patient.

A “decreased level” or “lower level” of a biomarker refers to a level that is quantifiably less than a predetermined value which may be a control value, e.g., the value found in normal subjects, or may also called the “cutoff value” and above the lower limit of quantitation (LLOQ). This determined “cutoff value” is specific for the algorithm and parameters related to patient sampling and treatment conditions.

A “higher level” or “elevated level” of a biomarker refers to a level that is quantifiably elevated relative to a predetermined value, which may be a control value, e.g., the value found in normal subjects or may also be called the “cutoff value.” This “cutoff value” is specific for the algorithm and parameters related to patient sampling and treatment conditions.

By “sample” or “patient's sample” is meant a specimen which is a cell, tissue, or fluid or portion thereof extracted, produced, collected, or otherwise obtained from a patient suspected to having or having presented with symptoms associated with SSc.

### Overview

Scleroderma or systemic sclerosis (SSc) is chronic disease of unknown cause characterized by diffuse fibrosis, degenerative changes, and vascular abnormalities in the skin, joints, and internal organs (especially the esophagus, lower GI tract, lung, heart, and kidney). Common symptoms include Raynaud's syndrome, polyarthralgia, dysphagia, heartburn, and swelling and eventually skin tightening and contractures of the fingers. SSc can develop as part of mixed connective tissue disease.

SSc is grouped among the putative autoimmune disorders: heredity and immunological mechanisms play a role. SSc-like symptoms are also provoked by exposure to certain chemicals; vinyl chloride, bleomycin, pentazocine (TALWIN®), epoxy and aromatic hydrocarbons, contaminated rapeseed oil, or 1-tryptophan (Merck Index, 2007 Ed.).

Systemic scleroderma can be divided into either “limited” cutaneous systemic sclerosis which affects only the forearms, hands, legs, feet, and face, or “diffuse” cutaneous systemic sclerosis which can affect almost any area of the body. SSc varies in severity and progression, ranging from generalized skin thickening with rapidly progressive and often fatal visceral involvement (SSc with diffuse scleroderma) to isolated skin involvement (often just the fingers and face) and slow progression (often several decades) before visceral disease develops. The latter form is termed limited cutaneous scleroderma or CREST syndrome (Calcinosis cutis, Raynaud's syndrome, Esophageal dysmotility, Sclerodactyly, Telangiectasias). In addition, SSc can overlap with other autoimmune rheumatic disorders, such as sclerodermatomyositis (tight skin and muscle weakness indistinguishable from polymyositis) and mixed connective tissue disease.

The pathophysiology of SSc involves vascular damage and activation of fibroblasts; collagen and other extracellular proteins in various tissues are overproduced. Thus, SSc may be accompanied by anticollagen antibodies and the presence of nucleolar and other nuclear antibodies, such as ANA and SCL-70 (SCL-70 antigen, topoisomerase-1, is a DNA-binding protein sensitive to nucleases).

Limited SSc patients (those with CREST syndrome) may have disease that is limited and nonprogressive for long periods; visceral changes including pulmonary hypertension caused by vascular disease of the lung, and a form of biliary cirrhosis eventually develop, but may not be severe.

Diffuse SSc patients eventually develop visceral complications, which are the usual causes of death. Prognosis is poor if cardiac, pulmonary, or renal manifestations are present early. Heart failure may be intractable. Ventricular ectopy, even if asymptomatic, increases the risk of sudden death. Acute renal insufficiency, if untreated, progresses rapidly and causes death within months.

Diffuse SSc patients may be further classified into 2 different subsets based on clinical parameters. Early progressive diffuse (EP) subjects are characterized by extensive skin and visceral involvement that typically progresses in a rapid fashion. Late improving diffuse (LI) subjects show improving skin often followed by stabilization of the disease.

No drug significantly influences the natural course of SSc overall, but various drugs are of value in treating specific symptoms or organ systems: NSAIDs for arthritis, corticosteroids for overt myositis or mixed connective tissue disease, but may predispose to renal crisis, immunosuppressives, such as methotrexate, azathioprine, and cyclophosphamide, may help pulmonary alveolitis, epoprostenol (prostacyclin) and bosentan and PDE-5 inhibitors (sildenafil, vardenafil, tadalafil) have been used for pulmonary hypertension, Ca channel blockers, such as nifedipine, or angiotensin receptor blockers, such as losartan, may help Raynaud's sydrome. IV infusions of prostaglandin E1 (alprostadil) or epoprostenol or sympathetic blockers can be used for digital ischemia. Reflux esophagitis is relieved by frequent small feedings, high-dose proton pump inhibitors, and sleeping with the head of the bed elevated. Esophageal strictures may require periodic dilation; gastroesophageal reflux may possibly require gastroplasty. Tetracycline or another broad-spectrum antibiotic can suppress overgrowth of intestinal flora and may alleviate malabsorption symptoms. Physiotherapy may help preserve muscle strength but is ineffective in preventing joint contractures. No treatment affects calcinosis. For acute renal crisis, prompt treatment with an ACE inhibitor can dramatically prolong survival. Blood pressure is usually, but not always, controlled. The mortality rate of renal crisis remains high. If end-stage renal disease develops, it may be reversible, but dialysis and transplantation may be necessary.

Diagnosis

The diagnosis of diffuse or limited SSc involves a clinical evaluation and tests for antinuclear antibodies (ANA), SCL-70 (topoisomerase I), and anticentromere antibodies. The clinical evaluation will include an assessment of the degree of skin involvement, typically using the modified Rodnan skin score (MRSS) as a standard outcome measure for skin disease in SSc and calculated by summation of skin thickness in 17 different body sites (total score=51). Severe organ involvement may be defined as the presence of any of the following: (1) in the kidney, scleroderma renal crisis; (2) in the heart, cardiomyopathy, symptomatic pericarditis, or an arrhythmia requiring treatment; (3) in the lung, pulmonary fibrosis on chest radiograph and a forced vital capacity of <55% of predicted; (4) in the GI tract, malabsorption, repeated episodes of pseudoobstruction, or severe problems requiring hyperalimentation; and (5) in the skin, a modified Rodnan skin score >40.

SSc should be considered in patients with Raynaud's syndrome, typical musculoskeletal or skin manifestations, or unexplained dysphagia, malabsorption, pulmonary fibrosis, pulmonary hypertension, cardiomyopathies, or conduction disturbances. Diagnosis can be obvious in patients with combinations of classic manifestations, such as Raynaud's syndrome, dysphagia, and tight skin However, in some patients, the diagnosis cannot be made clinically, and confirmatory laboratory tests can increase the probability of disease but do not rule it out.

ANA are present in ≧90%, often with an antinucleolar pattern. Antibody to centromeric protein (anticentromere antibody) occurs in the serum of a high proportion of patients with CREST syndrome and is detectable on the ANA. Patients with diffuse scleroderma are more likely than those with CREST to have anti-SCL-70 antibodies. Rheumatoid factor also is positive in 33% of patients.

If lung involvement is suspected, pulmonary function testing, chest CT, and echocardiography can begin to define its severity. Acute alveolitis is often detected by high-resolution chest CT.

Recent advances in technologies, such as proteomics, present pathologists with the challenge of integrating the new information generated with high-throughput methods with current diagnostic models based on clinicopathologic correlations and often with the inclusion of histopathological findings. Parallel developments in the field of medical informatics and bioinformatics provide the technical and mathematical methods to approach these problems in a rational manner providing new tools to the practitioner and pathologist or other medical specialists in the form multivariate and multidisciplinary diagnostic and prognostic models that are hoped to provide more accurate, individualized patient-based information. Evidence-based medicine (EBM) and medical decision analysis (MDA) are among the disciplines that use quantitative methods to assess the value of information and integrate so-called best evidence into multivariate models for the assessment of prognosis, response to therapy, and selection of laboratory tests that can influence individual patient care. The subject matter disclosed and claimed herein includes several aspects such as:
1. The use of serum to identify biomarkers associated with SSc patient population subsets.
2. The ability to correlate these biomarkers with SSc disease relevant clinical parameters
3. The use of these serum markers to predict response to therapy.

In order to define the markers useful in distinguishing SSc patients from normal subjects and subclassifying SSc patients as having limited or diffuse disease, serum from classified patients was analyzed for 92 different markers first and then 190 different markers using a multianalyte immunoassay panel or single analyte ELISA.

In addition to the other markers disclosed herein, the dataset markers may be selected from one or more clinical indicia, examples of which are age, race, gender, blood pressure, height and weight, body mass index, CRP concentration, tobacco use, heart rate, fasting insulin concentration, fasting glucose concentration, diabetes status, use of other medications, and specific functional or behavioral assessments, and/or radiological or other image-based assessments wherein a numerical values are applied to individual measures or an overall numerical score is generated. Clinical variables will typically be assessed and the resulting data combined in an algorithm with the described markers.

Prior to input into the analytical process, the data in each dataset is collected by measuring the values for each marker, usually in triplicate or in multiple triplicates. The data may be manipulated, for example, raw data may be transformed using standard curves, and the average of triplicate measurements used to calculate the average and standard deviation for each patient. These values may be transformed before being used in the models, e.g., log-transformed, Box-Cox transformed (see Box and Cox (1964) J. Royal Stat. Soc, Series B, 26:211-212; 1964), or other transformations known and practiced in the art. This data can then be input into the analytical process with defined parameters.

The quantitative data thus obtained related to the protein markers and other dataset components is then subjected to an analytic process with parameters previously determined using a learning algorithm, i.e., inputted into a predictive model, as in the examples provided herein (Examples 1 and 2). The parameters of the analytic process may be those disclosed herein or those derived using the guidelines described herein or known and practiced in the art. Learning algorithms, such as linear discriminant analysis, recursive feature elimination, a prediction analysis of microarray, logistic regression, CART, FlexTree, LART, random forest, MART, or another machine learning algorithm are applied to the appropriate reference or training data to determine the parameters for analytical processes suitable for a SSC classification.

The analytic process may set a threshold for determining the probability that a sample belongs to a given class. The probability preferably is at least 50%, or at least 60% or at least 70% or at least 80% or higher.

In other embodiments, the analytic process determines whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

In general, the analytical process will be in the form of a model generated by a statistical analytical method, such as a linear algorithm, a quadratic algorithm, a polynomial algorithm, a decision tree algorithm, a voting algorithm.

### Use of Reference/Training Datasets to Determine Parameters of Analytical Process

Using any suitable learning algorithm, an appropriate reference or training dataset is used to determine the parameters of the analytical process to be used for classification, i.e., develop a predictive model.

The reference, or training dataset, to be used will depend on the desired PsA classification to be determined, e.g., responder or non-responder. The dataset may include data from two, three, four, or more classes.

For example, to use a supervised learning algorithm to determine the parameters for an analytic process used to predict response to SSc therapy agent, a dataset comprising control and diseased samples is used as a training set. Alternatively, a supervised learning algorithm is to be used to develop a predictive model for SSc therapy.

### Statistical Analysis

The following are examples of the types of statistical analysis methods that are available to one of skill in the art to aid in the practice of the disclosed methods. The statistical analysis may be applied for one or both of two tasks. First, these and other statistical methods may be used to identify preferred subsets of the markers and other indicia that will form a preferred dataset. In addition, these and other statistical methods may be used to generate the analytical process that will be used with the dataset to generate the result. Several of statistical methods presented herein or otherwise available in the art will perform both of these tasks and yield a model that is suitable for use as an analytical process for the practice of the methods disclosed herein.

In a specific embodiment, biomarkers and their corresponding features (e.g., expression levels or serum levels) are used to develop an analytical process, or plurality of analytical processes, that discriminate between classes of patients, e.g., those with diffuse disease, those with limited disease and normal non-diseased subjects. Once an analytical process has been built using these exemplary data analysis algorithms or other techniques known in the art, the analytical process can be used to classify a test subject into one of the two or more phenotypic classes (e.g., a patient predicted to require treatment for diffuse SSc or a patient predicted to required treatment for limited SSc, or those subjects not requiring treatment for SSc). This is accomplished by applying the analytical process to a marker profile obtained from the test subject. Such analytical processes, therefore, have value as diagnostic indicators.

In one aspect, the disclosed methods provide for the evaluation of a marker profile from a test subject to marker profiles obtained from a training population. In some embodiments, each marker profile obtained from subjects in the training population, as well as the test subject, comprises a feature for each of a plurality of different markers. In further embodiments, this comparison is accomplished by (i) developing an analytical process using the marker profiles from the training population and (ii) applying the analytical process to the marker profile from the test subject. As such, the analytical process applied in some embodiments of the methods disclosed herein is used to determine whether a test SSc patient is predicted to respond to treatment.

Thus, in some embodiments, the result in the above-described binary decision situation has four possible outcomes: (i) a true responder, where the analytical process indicates that the subject will be a responder to therapy and the subject responds to therapy during the definite time period (true positive, TP); (ii) false responder, where the analytical process indicates that the subject will be a responder to therapy and the subject does not respond to therapy during the definite time period (false positive, FP); (iii) true non-responder, where the analytical process indicates that the subject will not be a responder to therapy and the subject does not respond to therapy during the definite time period (true negative, TN); or (iv) false non-responder, where the analytical process indicates that the patient will not be a responder to therapy and the subject does in fact respond to therapy during the definite time period (false negative, FN).

Relevant data analysis algorithms for developing an analytical process include, but are not limited to, discriminant analysis including linear, logistic, and more flexible discrimination techniques (see, e.g., Gnanadesikan, 1977, Methods for Statistical Data Analysis of Multivariate Observations, New York: Wiley 1977, which is hereby incorporated by reference herein in its entirety); tree-based algorithms such as classification and regression trees (CART) and variants (see, e.g., Breiman, 1984, Classification and Regression Trees, Belmont, Calif.; Wadsworth International Group); generalized additive models (see, e.g., Tibshirani, 1990, Generalized Additive Models, London: Chapman and Hall); and neural networks (see, e.g., Neal, 1996, Bayesian Learning for Neural Networks, New York: Springer-Verlag; and Insua, 1998); Feedforward neural networks for nonparametric regression In: Practical Nonparametric and Semiparametric Bayesian Statistics, pp. 181-194, New York: Springer. These references are hereby incorporated by reference in their entirety.

In a specific embodiment, a data analysis algorithm of the invention comprises Classification and Regression Tree (CART), Multiple Additive Regression Tree (MART), Prediction Analysis for Microarrays (PAM) or Random Forest analysis. Such algorithms classify complex spectra from biological materials, such as a blood sample, to distinguish subjects as normal or as possessing biomarker expression levels characteristic of a particular disease state. In other embodiments, a data analysis algorithm of the invention comprises ANOVA and nonparametric equivalents, linear discriminant analysis, logistic regression analysis, nearest neighbor classifier analysis, neural networks, principal component analysis, quadratic discriminant analysis, regression classifiers and support vector machines.

While such algorithms may be used to construct an analytical process and/or increase the speed and efficiency of the application of the analytical process and to avoid investigator bias, one of ordinary skill in the art will realize that a computer-based device is not required to carry out the methods of using the classification models of the present invention.

Marker Sets for Systemic Sclerosis Analysis In one aspect of the present invention, the analyses of markers in patients diagnosed with SSc was focused on defining those markers that can be used to distinguish a SSc patient from a subject not afflicted with SSc. In another aspect, the invention provides a second set of markers that can be used to distinguish a patient having limited SSc from a patient having diffuse SSc. In yet another aspect, the invention provides a set of markers that can be used to distinguish a subgroup of diffuse SSc patients from other patients diagnosed with SSc.

The specific examples described herein for generating an algorithm useful for diagnosis of a SSc patient indicate that multiple markers are correlative of processes involved in the pathophysiology of SSc and the quantitative interpretation of each particular biomarker in diagnosing or predicting response to therapy has not been heretofore well established. The present invention demonstrates that an analytical method can be generated using a sampling of patient data based on specific markers defined. In one method of using the markers of the invention, a computer assisted device is used to capture patient data and perform the necessary analysis. In another aspect, the computer assisted device or system may use the data presented herein as a “training data set” in order to generate the classifier information required to apply the predictive analysis.

### Instruments, Reagents and Kits for Performing the Analysis

The measurement of serum biomarkers for predicting response of a diagnosed SSc patient to therapy may be performed in a clinical or research laboratory or a centralized laboratory in a hospital or non-hospital location using standard immunochemical and biophysical methods as described herein. The marker quantitation may be performed at the same time as e.g., other standard measures such as WBC count, platelets, and ESR. The analysis may be performed individually or in batches using commercial kits, or using multiplexed analysis on individual patient samples.

In one aspect of the invention, individual and sets of reagents are used in one or more steps to determine relative or absolute amounts of a biomarker, or panel or biomarkers, in a patient's sample. The reagents may be used to capture the biomarker, such as an antibody immunospecific for a biomarker, which forms a ligand biomarker pair detectable by an indirect measurement, such as enzyme-linked immunospecific assay. Either single analyte EIA or multiplexed analysis can be performed. Multiplexed analysis is a technique by which multiple, simultaneous EIA-based assays can be performed using a single serum sample. One platform useful to quantify large numbers of biomarkers in a very small sample volume is the xMAP® technology used by Rules Based Medicine in Austin, Tex. (owned by the Luminex Corporation), which performs up to 100 multiplexed, microsphere-based assays in a single reaction vessel by combining optical classification schemes, biochemical assays, flow cytometry and advanced digital signal processing hardware and software. In the technology, multiplexing is accomplished by assigning each analyte-specific assay a microsphere set labeled with a unique fluorescence signature. Multiplexed assays are analyzed in a flow device that interrogates each microsphere individually as it passes through a red and green laser. Alternatively, methods and reagents are used to process the sample for detection and possible quantitation using a direct physical measurement, such as mass, charge, or a combination, such as by SELDI. Quantitative mass spectrometric multiple reaction monitoring assays have also been developed such as those offered by NextGen Sciences (Ann Arbor, Mich.).

According to one aspect of the invention, therefore, the detection of biomarkers for evaluation of SSc status entails contacting a sample from a subject with a substrate, e.g., a probe, having capture reagent thereon, under conditions that allow binding between the biomarker and the reagent, and then detecting the biomarker bound to the adsorbent by a suitable method. One method for detecting the marker is gas phase ion spectrometry, for example, mass spectrometry. Other detection paradigms that can be employed to this end include optical methods, electrochemical methods (voltometry, amperometry or electrochemiluminescent techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry), and enzyme-coupled colorimetric or fluorescent methods.

Specimens from patients may require processing prior to applying the detecting method to the processed specimen or sample such as but not limited to methods to concentrate, purify, or separate the marker from other components of the specimen. For example a blood sample is typically allowed to clot followed by centrifugation to produce serum or treated with an anticoagulant and the cellular components and platelets removed prior to being subjected to methods of detecting analyte concentration. Alternatively, the detecting may be accomplished by a continuous processing system which may incorporate materials or reagents to accomplish such concentrating, separating or purifying steps. In one embodiment, the processing system includes the use of a capture reagent. One type of capture reagent is a “chromatographic adsorbent,” which is a material typically used in chromatography. Chromatographic adsorbents include, for example, ion exchange materials, metal chelators, immobilized metal chelates, hydrophobic interaction adsorbents, hydrophilic interaction adsorbents, dyes, simple biomolecules (e.g., nucleotides, amino acids, simple sugars and fatty acids), mixed mode adsorbents (e.g., hydrophobic attraction/electrostatic repulsion adsorbents). A “biospecific” capture reagent is a capture reagent that is a biomolecule, e.g., a nucleotide, a nucleic acid molecule, an amino acid, a polypeptide, a polysaccharide, a lipid, a steroid or a conjugate of these (e.g., a glycoprotein, a lipoprotein, a glycolipid). In certain instances the biospecific adsorbent can be a macromolecular structure such as a multiprotein complex, a biological membrane or a virus. Illustrative biospecific adsorbents are antibodies, receptor proteins, and nucleic acids. A biospecific adsorbent typically has higher specificity for a target analyte than a chromatographic adsorbent.

The detection and quantitation of the biomarkers according to the invention can thus be enhanced by using certain selectivity conditions, e.g., adsorbents or washing solutions. A wash solution refers to an agent, typically a solution, which is used to affect or modify adsorption of an analyte to an adsorbent surface and/or to remove unbound materials from the surface. The elution characteristics of a wash solution can depend, for example, on pH, ionic strength, hydrophobicity, degree of chaotropism, detergent strength, and temperature.

In one aspect of the present invention, a sample is analyzed in a multiplexed manner meaning that the processing of markers from a patient samples occurs nearly simultaneously. In one aspect, the sample is contacted by a substrate comprising multiple capture reagents representing unique specificity. The capture reagents are commonly immunospecific antibodies or fragments thereof. The substrate may be a single component such as a “biochip,” a term that denotes a solid substrate, having a generally planar surface, to which a capture reagent(s) is attached, or the capture reagents may be segregated among a number of substrates, as for example bound to individual spherical substrates (beads). Frequently, the surface of a biochip comprises a plurality of addressable locations, each of which has the capture reagent bound there. A biochip can be adapted to engage a probe interface and, hence, function as a probe in gas phase ion spectrometry preferably mass spectrometry. Alternatively, a biochip of the invention can be mounted onto another substrate to form a probe that can be inserted into the spectrometer. In the case of the beads, the individual beads may be partitioned or sorted after exposure to the sample for detection.

A variety of biochips are available for the capture and detection of biomarkers, in accordance with the present invention, from commercial sources such as Ciphergen Biosystems (Fremont, Calif.), Perkin Elmer (Packard BioScience Company (Meriden Conn.), Zyomyx (Hayward, Calif.), and Phylos (Lexington, Mass.), GE Healthcare, Corp. (Sunnyvale, Calif.). Exemplary of these biochips are those described in U.S. Pat. No. 6,225,047, supra, and No. 6,329,209 (Wagner et al.), and in WO 99/51773 (Kuimelis and Wagner), WO 00/56934 (Englert et al.) and particularly those which use electrochemical and electrochemiluminescence methods of detecting the presence or amount of an analyte marker in a sample such as those multi-specific, multi-array taught in Wohlstadter et al., WO98/12539 and U.S. Pat. No. 6,066,448.

A substrate with specific capture and/or detection reagents is contacted with the sample, containing e.g., serum, for a period of time sufficient to allow the biomarker that may be present to bind to the reagent. In one embodiment of the invention, more than one type of substrate with specific capture or detection reagents thereon is contacted with the biological sample. After the incubation period, the substrate is washed to remove unbound material. Any suitable washing solutions can be used; preferably, aqueous solutions are employed.

Biomarkers bound to the substrates are to be detected after desorption directly by using a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined. Such methods may be used to discovery biomarkers and, in some instances for quantitation of biomarkers.

In another embodiment, the method of the invention is a microfluidic device capable of miniaturized liquid sample handling and analysis device for liquid phase analysis as taught in, for example, U.S. Pat. No. 5,571,410 and U.S. RE36350, useful for detecting and analyzing small and/or macromolecular solutes in the liquid phase, optionally, employing chromatographic separation means, electrophoretic separation means, electrochromatographic separation means, or combinations thereof. The microfluidic device or “microdevice” may comprise multiple channels arranged so that analyte fluid can be separated, such that biomarkers may be captured, and, optionally, detected at addressable locations within the device (U.S. Pat. No. 5,637,469; U.S. Pat. No. 6,046,056 and U.S. Pat. No. 6,576,478).

Data generated by detection of biomarkers can be analyzed with the use of a programmable digital computer. The computer program analyzes the data to indicate the number of markers detected and the strength of the signal. Data analysis can include steps of determining signal strength of a biomarker and removing data deviating from a predetermined statistical distribution. For example, the data can be normalized relative to some reference. The computer can transform the resulting data into various formats for display, if desired, or further analysis.

### Artificial Neural Network

In some embodiments, a neural network is used. A neural network can be constructed for a selected set of markers. A neural network is a two-stage regression or classification model. A neural network has a layered structure that includes a layer of input units (and the bias) connected by a layer of weights to a layer of output units. For regression, the layer of output units typically includes just one output unit. However, neural networks can handle multiple quantitative responses in a seamless fashion.

In multilayer neural networks, there are input units (input layer), hidden units (hidden layer), and output units (output layer). There is, furthermore, a single bias unit that is connected to each unit other than the input units. Neural networks are described in Duda et al., 2001, Pattern Classification, Second Edition, John Wiley &amp; Sons, Inc., New York; and Hastie et al., 2001, The Elements of Statistical Learning, Springer-Verlag, New York.

The basic approach to the use of neural networks is to start with an untrained network, present a training pattern, e.g., marker profiles from patients in the training data set, to the input layer, and to pass signals through the net and determine the output, e.g., the prognosis of the patients in the training data set, at the output layer. These outputs are then compared to the target values, e.g., actual outcomes of the patients in the training data set; and a difference corresponds to an error. This error or criterion function is some scalar function of the weights and is minimized when the network outputs match the desired outputs. Thus, the weights are adjusted to reduce this measure of error. For regression, this error can be sum-of-squared errors. For classification, this error can be either squared error or cross-entropy (deviation). See, e.g., Hastie et al., 2001, The Elements of Statistical Learning, Springer-Verlag, New York.

Three commonly used training protocols are stochastic, batch, and on-line. In stochastic training, patterns are chosen randomly from the training set and the network weights are updated for each pattern presentation. Multilayer nonlinear networks trained by gradient descent methods such as stochastic back-propagation perform a maximum-likelihood estimation of the weight values in the model defined by the network topology. In batch training, all patterns are presented to the network before learning takes place. Typically, in batch training, several passes are made through the training data. In online training, each pattern is presented once and only once to the net.

In some embodiments, consideration is given to starting values for weights. If the weights are near zero, then the operative part of the sigmoid commonly used in the hidden layer of a neural network (see, e.g., Hastie et al., 2001, The Elements of Statistical Learning, Springer-Verlag, New York) is roughly linear, and hence the neural network collapses into an approximately linear model. In some embodiments, starting values for weights are chosen to be random values near zero. Hence the model starts out nearly linear, and becomes nonlinear as the weights increase. Individual units localize to directions and introduce nonlinearities where needed. Use of exact zero weights leads to zero derivatives and perfect symmetry, and the algorithm never moves. Alternatively, starting with large weights often leads to poor solutions.

Since the scaling of inputs determines the effective scaling of weights in the bottom layer, it can have a large effect on the quality of the final solution. Thus, in some embodiments, at the outset all expression values are standardized to have mean zero and a standard deviation of one. This ensures all inputs are treated equally in the regularization process, and allows one to choose a meaningful range for the random starting weights. With standardization inputs, it is typical to take random uniform weights over the range sigma −0.7, +0.7 sigma.

A recurrent problem in the use of networks having a hidden layer is the optimal number of hidden units to use in the network. The number of inputs and outputs of a network are determined by the problem to be solved. For the methods disclosed herein, the number of inputs for a given neural network can be the number of markers in the selected set of markers.

The number of outputs for the neural network will typically be just one: yes or no. However, in some embodiment more than one output is used so that more than two states can be defined by the network.

Software used to analyze the data can include code that applies an algorithm to the analysis of the signal to determine whether the signal represents a peak in a signal that corresponds to a biomarker according to the present invention. The software also can subject the data regarding observed biomarker signals to classification tree or ANN analysis, to determine whether a biomarker or combination of biomarker signals is present that indicates patient's disease diagnosis or status.

Thus, the process can be divided into the learning phase and the classification phase. In the learning phase, a learning algorithm is applied to a data set that includes members of the different classes that are meant to be classified, for example, data from a plurality of samples from patients diagnosed as SSc and samples for normal control subjects; or patients diagnosed with limited SSc and patients diagnosed with diffuse SSc; or patients diagnosed with diffuse SSc and SSc patients know to have organ involvement. The methods used to analyze the data include, but are not limited to, artificial neural network, support vector machines, genetic algorithm and self-organizing maps, and classification and regression tree (CART) analysis. These methods are described, for example, in WO01/31579, May 3, 2001 (Barnhill et al.); WO02/06829, Jan. 24, 2002 (Hitt et al.) and WO02/42733, May 30, 2002 (Paulse et al.). The learning algorithm produces a classifying algorithm keyed to elements of the data, such as particular markers and specific concentrations of markers, usually in combination, that can classify an unknown sample into one of the two classes, e.g., SSc or normal, responder on non-responder. The classifying algorithm is ultimately used for either diagnostic or predictive testing.

Software, both freeware and proprietary software, is readily available to analyze patterns in data, and to devise additional patterns with any predetermined criteria for success.

### Kits

In another aspect, the present invention provides kits for capable of determining the concentrations of the markers or marker sets useful in distinguishing whether a subject is to be diagnosed with SSc, whether a patient diagnosed with SSc is classified as having limited or diffuse disease, or whether a patient diagnosed with SSc is among the subset of patients with diffuse disease classifiable distinguished form other diagnosed SSc patients with diffuse or limited disease. The kits comprise the tools and reagents useful in detecting and quantifying the presence of serum markers and combinations of markers that are differentially present in SSc patients.

In one aspect, the kit contains a means for collecting a sample, such as a lance or piercing tool for causing a “stick” through the skin The kit may, optionally, also contain a probe, such as a capillary tube, or blood collection tube for collecting blood from the stick.

In one embodiment, the kit comprises a substrate having one or more biospecific capture reagents for binding a marker according to the invention. The kit may include more than type of biospecific capture reagents, each present on the same or a different substrate.

In a further embodiment, such a kit can comprise instructions for suitable operational parameters in the form of a label or separate insert. For example, the instructions may inform a consumer how to collect the sample or how to empty or wash the probe. In yet another embodiment the kit can comprise one or more containers with biomarker samples, to be used as standard(s) for calibration.

In the method of using the method of the invention for diagnosing or classifying patient with SSC or for monitoring the response to therapy, blood or other fluid is acquired from the patient prior to therapy and at specified periods after therapy is initiated. The blood may be processed to extract a serum or plasma fraction or may be used whole. The blood or serum samples may be diluted, for example 1:2, 1:5, 1:10, 1:20, 1:50, or 1:100, or used undiluted. In one format, the serum or blood sample is applied to a prefabricated test strip or stick and incubated at room temperature for a specified period of time, such as 1 min, 5 min, 10 min, 15, min, 1 hour, or longer. After the specified period of time for the assay; the samples and the result are readable directly from the strip. For example, the results appear as varying shades of colored or gray bands, indicating a concentration range of one or more markers. The test strip kit will provide instructions for interpreting the results based on the relative concentrations of the one or more markers. Alternatively, a device capable of detecting the color saturation of the marker detection system on the strip can be provided, which device may optionally provide the results of the test interpretation based on the appropriate diagnostic algorithm for that series of markers.

### Methods of Using the Invention

The invention provides a method of stratifying or classifying patients suspected of or having been clinically diagnosed with SSc. The biomarkers of the invention may be further used to monitor or predict responsiveness to therapy with an anti-SSC agent. An anti-SSc agent may be an anti-inflammatory, such as penicillamine, or anti-immune mediator such as a TNFalpha antagonist, or a nutrient or anti-nutrient, or modality such as heat or penetrating radiant energy, or some combination of agents and/or modalities. By analyzing detected biomarkers in a patient diagnosed with SSc by an experienced professional using subjective and objective criteria, the patient may be further classified as having limited disease or having diffuse disease.

In the method of the invention for diagnosing or subclassifying SSc prior to the recommendation or initiation of therapy, at a “baseline visit,” a baseline or “Week 0” sample is acquired from the subject. The sample may be any tissue which can be evaluated for the biomarkers associated with the method of the invention. In one embodiment the sample is a fluid selected from the group consisting of a fluid selected from the group consisting of blood, serum, plasma, urine, semen and stool. In a particular embodiment, the sample is a serum sample which is obtained from patient's blood drawn by a standard method of direct venipuncture or via an intravenous catheter.

In addition, at the baseline visit, information on patient's demographics and history of disease symptoms may be recorded on a standardized form or case report form. Data such as time since patient's diagnosis, previous treatment history, concomitant medications, and other clinical test results will be recorded.

The results of the biomarker analysis for at least the markers described herein; reported as concentrations in units of weight, particles, molecules, or fragments thereof, in the patient's sample will be compared to a normal standard or historical values for normal subjects using the same units. The ratio of the concentration marker in the patient's sample to the concentration in the normal standard or the historic value for normal subjects is calculated and the values for the ratios of sample to standard are tabulated or otherwise recorded so that it may be recognized whether the value for the ratio for each individual marker is greater than 2. When the ratios of the concentrations of the markers versus the concentration in the normal standard or the historic value for normal subjects are greater than 2, the patient is likely to be suffering from SSc.

For patients suspected of having or having been diagnosed with scleroderma or SSc, the results of the biomarker analysis for at least the markers IL13, IL17, IgE, and GST reported as concentrations in units of weight, particles, molecules, or fragments thereof in the patient's sample will be compared to historical values for the same marker using the same units in serum from patients previously diagnosed with limited SSc or diffuse SSc. The ratio of the concentration marker in the patient's sample to the concentration in the historical values for the same marker using the same units in serum from patients previously diagnosed with limited SSc or diffuse SSc is calculated and the values for the ratios of sample to standard are tabulated or otherwise recorded so that it may be recognized with the ratio or IL17 is less than 1 when compared to the standard or values for patients having limited SSc and greater than 1 when compared to standard or values from patients having diffuse SSc; and, in addition, if the ratio of IL13 concentration to standard or value for limited SSc is recognized as greater than 1, or is less than 1 when compared to diffuse SSc and, in addition, if the ratio of IgE concentration to standard or value from patients with diffuse SSc is recognized as greater than 1, or less than 1 when compared to the standard or value from patients with limited SSc; and, in addition, if the ratio of GST concentration to standard or value from patients with diffuse SSc is recognized as less than 1, or when compared to the standard or value from patients with limited SSc is greater than 1; then the patient is likely suffering from limited SSc.

For patients suspected of having or having been diagnosed with diffuse SSc, the results of the biomarker analysis for at least the markers VEGF, fibrinogen, IL-13, IL-17 as well as CXCL5, CCL2, CCL5, CCL11, BDNF, MPO, and EGF reported as concentrations in units of weight, particles, molecules, or fragments thereof; in the patient's sample will be compared to historical values for the same marker using the same units in serum from patients previously diagnosed with limited SSc and diffuse SSc to further distinguish a subset of patients with diffuse SSc.

The patient is scheduled for subsequent visits, such as a Week 8, Week 12, Week 14, Week 28, etc. visit for the purposes of performing assessment of disease using the such criteria as set forth by, e.g., the physician or an expert panel, and for the acquisition of patient samples for biomarker evaluation.

At any or the above times prior to, during, or following treatment, other parameters and markers may be assessed in the patient's sample or other fluid or tissue samples acquired from the patient. These may include standard hematological parameters, such as hemoglobin content, hematocrit, red cell volume, mean red cell diameter, erythrocyte sedimentation rate (ESR), and the like.

The medical professional's clinical judgment of response should not be negated by the test result. However, the test could aid in making the decision to continue or discontinue treatment with golimumab. In a test in which the prediction model (algorithm) has 90% sensitivity and 60% specificity, where 50% of the patients display a clinical response and 50% do not display assessment scores or evaluations consistent with a clinical response. This would mean: of the responders, 45% would be identified correctly as responders (5 would be reported as likely non-responders) and 30% or non-responders would be identified correctly as non-responders (20% would be classified as likely responders). Thus, overall benefit is that 60% of all true non-responders could be spared an unnecessary therapy or discontinued from therapy at an early time point (Week 4). The 5% false-negative “responders” (identified as likely non-responders) would have been treated, and as with all patients, their response would be judged clinically before making the decision to continue or discontinue treatment at Week 14 or later. The 20% false-negative “non-responders” (identified as possible responders) would have to be judged clinically, and would take the usual time to make the decision to discontinue treatment.

### Example 1

### Sample Collection and Analysis

In order to define the markers useful in distinguishing SSc patient subsets, serum from a Biobank of SSc serum samples (Thomas Jefferson University) was used. The SSc serum cohort consisted of data from 38 subjects with diffuse SSc and 36 subjects with limited SSc. The available clinical parameters included age of onset, peak skin score, lung involvement, peripheral white blood cell count. The serum values for all analytes were compared to data pooled from 160 healthy normal subjects (Centocor internal data).

The sera were analyzed for biomarkers using commercially available assays employing either a multiplex analysis performed by Rules Based Medicine (Austin, Tex.) or single analyte ELISA. All samples were stored at −80° C. until tested. The samples were thawed at room temperature, vortexed, spun at 13,000×g for 5 minutes for clarification and 150 uL was removed for antigen analysis into a master microtiter plate. Analysis was performed in a Luminex 100 instrument and the resulting data stream was interpreted using data analysis software from OmniViz and NCSS. For each multiplex, both calibrators and controls were run.

Testing results were determined first for the high, medium and low controls for each multiplex to ensure proper assay performance. Unknown values for each of the analytes localized in a specific multiplex were determined using 4 and 5 parameter, weighted and non-weighted curve fitting algorithms included in the data analysis package.

TABLE 1

| | | Swiss-Prot |
|---|---|---|
| Protein Biomarker | Units | Accession # |
| ACE (CD143) Angiotensin Converting Enzyme | ng/ml | P12821 |
| ACTH (Adrenocorticotropic Hormone) | ng/mL | P01189 |
| Adiponectin | ug/mL | Q15848 |
| Agouti-Related Protein (AgRP) | pg/mL | O00253 |
| Alpha 1-Antichymotrypsin | ug/ml | P01011 |
| Alpha-1 Antitrypsin | mg/mL | P07758 |
| Alpha-1-Microglobulin | ug/ml | P02760 |
| Alpha-2 Macroglobulin | mg/mL | P01023 |
| Alpha-Fetoprotein | ng/mL | P02771 |
| Amphiregulin | pg/mL | n/a |
| Angiopoietin 2 (ANG-2) | ng/mL | O15123 |
| Angiotensinogen | ng/mL | P01019 |
| Apolipoprotein A1 | mg/mL | P02647 |
| Apolipoprotein A2 | ng/ml | P02652 |
| Apolipoprotein A-IV | ug/ml | n/a |
| Apolipoprotein B | ug/ml | P04114 |
| Apolipoprotein CI | ng/ml | P02654 |
| Apolipoprotein CIII | ug/mL | P02656 |
| Apolipoprotein D | ug/ml | P05090 |
| Apolipoprotein E | ug/ml | P02649 |
| Apolipoprotein H | ug/mL | P02749 |
| AXL | ng/mL | P30530 |
| Beta-2 Microglobulin | ug/mL | P01884 |
| Betacellulin | pg/mL | P35070 |
| B-Lymphocyte Chemoattractant (BLC) | pg/ml | O43927 |
| BMP-6 | ng/mL | P22004 |
| Brain-Derived Neurotrophic Factor | ng/mL | P23560 |
| C Reactive Protein | ug/mL | P02741 |
| Calbindin | ng/ml | P05937 |
| Calcitonin | pg/mL | P01258 |
| Cancer Antigen 125 | U/mL | Q14596 |
| Cancer Antigen 19-9 | U/mL | Q9BXJ9 |
| Carcinoembryonic Antigen | ng/mL | P78448 |
| CD40 | ng/mL | P25942 |
| CD40 Ligand | ng/mL | P29965 |
| CD5L | ng/ml | O43866 |
| CgA | ng/mL | P01215 |
| Ciliary Neurotrophic Factor (CNTF) | pg/mL | P26441 |
| Clusterin (Apo J) | ug/ml | P10909 |
| Complement 3 | mg/mL | P01024 |
| Complement Factor H | ug/ml | P08603 |
| Connective Tissue Growth Factor (CTGF) | ng/ml | P29279 |
| Cortisol | ng/ml | H02AB09 |
| C-peptide | ng/ml | P01308 |
| Creatine Kinase-MB | ng/mL | P12277 |
| Cystatin C | ng/ml | P01034 |
| EGF | pg/mL | P01133 |
| EGF-R | ng/mL | P00533 |
| ENA-78 | ng/mL | P42830 |
| Endothelin-1 | pg/mL | P05305 |
| EN-RAGE | ng/mL | P80511 |
| Eotaxin | pg/mL | P51671 |
| Eotaxin-3 | pg/mL | Q9Y258 |
| Epiregulin | pg/mL | O14944 |
| Erythropoietin | pg/mL | P01588 |
| E-Selectin | ng/mL | P16581 |
| Factor VII | ng/mL | P08709 |
| FAS | ng/mL | P25445 |
| Fas-Ligand | pg/mL | P48023 |
| Fatty Acid Binding Protein | ng/mL | P05413 |
| Ferritin | ng/mL | P02792 |
| Fetuin A | ug/ml | P02794 |
| FGF basic | pg/mL | P09038 |
| FGF-4 | pg/mL | P08620 |
| Fibrinogen | mg/mL | P02671 |
| FSH (Follicle Stimulation Hormone) | ng/ml | P01225 |
| Gamma-Interferon-induced-Monokine | pg/ml | Q07325 |
| G-CSF | pg/mL | P09919 |
| GLP-1 total (Glucagon-like Peptide-1, total) | pg/ml | P43220 |
| Glucagon | pg/ml | P01275 |
| Glutathione S-Transferase alpha (GST-alpha) | ng/ml | P08263 |
| GM-CSF | pg/mL | P04141 |
| GRO-alpha | pg/mL | P09341 |
| Growth Hormone | ng/mL | P01241 |
| Haptoglobin | mg/mL | P00738 |
| HB-EGF | pg/mL | Q99075 |
| HCC-4 | ng/mL | O15467 |
| Heat Shock Protein 60 | ng/ml | P10809 |
| Hepatocyte Growth Factor (HGF) | ng/mL | P14210 |
| I-309 | pg/mL | P22362 |
| ICAM-1 | ng/mL | P05362 |
| IFN-gamma | pg/mL | P01579 |
| IgA | mg/mL | na |
| IgE | ng/mL | na |
| IGF BP-2 | ng/mL | P18065 |
| IGF-1 | ng/mL | P01343 |
| IgM | mg/mL | na |
| IL-10 | pg/mL | P22301 |
| IL-11 | pg/mL | P20809 |
| IL-12p40 | ng/mL | P29460 |
| IL-12p70 | pg/mL | P29459 |
| IL-13 | pg/mL | P35225 |
| IL-15 | ng/mL | P40933 |
| IL-16 | pg/mL | Q14005 |
| IL-17E | pg/mL | Q9H293 |
| IL-18 | pg/mL | Q14116 |
| IL-1alpha | ng/mL | P01583 |
| IL-1beta | pg/mL | P01584 |
| IL-1ra | pg/mL | Q9UBH0 |
| IL-2 | pg/mL | P01585 |
| IL-3 | ng/mL | P08700 |
| IL-4 | pg/mL | P05112 |
| IL-5 | pg/mL | P05113 |
| IL-6 | pg/mL | P05231 |
| IL-6 Receptor | ng/mL | P08887 |
| IL-7 | pg/mL | P13232 |
| IL-8 | pg/mL | P10145 |
| Insulin | uIU/mL | P01308 |
| IP-10 (Inducible Protein-10) | pg/ml | P02778 |
| Kidney Injury Molecule-1 (KIM-1) | ng/ml | Q96D42 |
| Leptin | ng/mL | P41159 |
| LH (Luteinizing Hormone) | ng/ml | P01229 |
| Lipoprotein (a) | ug/mL | P08519 |
| LOX-1 | ng/mL | P78380 |
| Lymphotactin | ng/mL | P47992 |
| MCP-1 | pg/mL | P13500 |
| MCP-2 | pg/ml | P80075 |
| MCP-3 | pg/mL | P80098 |
| MCP-4 | pg/ml | Q99616 |
| M-CSF | ng/mL | P09603 |
| MDA-LDL | ng/mL | |
| MDC | pg/mL | O00626 |
| MIF | ng/mL | P14174 |
| MIP-1alpha | pg/mL | P10147 |
| MIP-1beta | pg/mL | P13236 |
| MIP-3 alpha | pg/ml | P78556 |
| MMP-1 | ng/ml | P03956 |
| MMP10 | ng/ml | P09238 |
| MMP-2 | ng/mL | P08253 |
| MMP-3 | ng/mL | P08254 |
| MMP7 | ng/ml | P09237 |
| MMP-9 | ng/mL | P14780 |
| MMP9 (Total) | ng/ml | P14780 |
| Myeloid Progenitor Inhibitory Factor 1 | ng/mL | P55773 |
| Myeloperoxidase | ng/mL | P05164 |
| Myoglobin | ng/mL | P02144 |
| Neutrophil Gelatinase-Associated Lipocalin | ng/ml | P80188 |
| (NGAL) | | |
| NGFb | ng/mL | P01138 |
| NrCAM | ng/mL | Q92823 |
| NT-proBNP | pg/ml | P16860 |
| Osteopontin | ng/ml | P10451 |
| PAI-1 | ng/mL | P05121 |
| Pancreatic polypeptide | pg/ml | P01298 |
| PAPP-A | mIU/mL | Q13219 |
| PDGF-BB | pg/ml | P01127 |
| PLGF | pg/ml | |
| Progesterone | ng/ml | |
| Proinsulin, Intact | pM | P01308 |
| Proinsulin, Total | pM | P01308 |
| Prolactin | ng/ml | P01236 |
| Prostate Specific Antigen, Free | ng/mL | P07288 |
| Prostatic Acid Phosphatase | ng/mL | P15309 |
| Protein S | ug/ml | P07225 |
| Pulmonary and Activation-Regulated | ng/mL | P55774 |
| Chemokine (PARC) | | |
| PYY | pg/mL | P55774 |
| RANTES | ng/mL | P13501 |
| Resistin | ng/ml | Q9HD89 |
| S100b | ng/mL | P04271 |
| Secretin | ng/mL | P09683 |
| Serum Amyloid P | ug/mL | P02743 |
| SGOT | ug/mL | P17174 |
| SHBG | nmol/L | P04278 |
| SOD | ng/mL | P08294 |
| Sortilin | ng/mL | Q99523 |
| sRAGE | ng/mL | Q15109 |
| Stem Cell Factor | pg/mL | P21583 |
| Tamm-Horsfall Protein (THP) | ug/ml | P07911 |
| Tenascin C | ng/mL | P24821 |
| Testosterone | ng/ml | |
| TGF-alpha | pg/mL | P01135 |
| TGF-beta 3 | pg/mL | P10600 |
| Thrombomodulin | ng/ml | P07204 |
| Thrombopoietin | ng/mL | P40225 |
| Thrombospondin-1 | ng/mL | P07996 |
| Thymus-Expressed Chemokine (TECK) | ng/mL | O15444 |
| Thyroid Stimulating Hormone | uIU/mL | P01215 |
| Thyroxine Binding Globulin | ug/mL | P05543 |
| TIMP-1 | ng/mL | P01033 |
| Tissue Factor | ng/mL | P13726 |
| TNF RII | ng/mL | Q92956 |
| TNF-alpha | pg/mL | P01375 |
| TNF-beta | pg/mL | P01374 |
| TRAIL-R3 | ng/mL | O14763 |
| Transferrin | mg/dl | P02787 |
| Trefoil Factor 3 (TFF3) | ug/ml | Q07654 |
| TTR (prealbumin) | mg/dl | P02766 |
| VCAM-1 | ng/mL | P19320 |
| VEGF | pg/mL | P15692 |
| Vitronectin | ug/ml | P04004 |
| von Willebrand Factor | ug/mL | P04275 |

Each of the 92 biomarkers in the initial panel has an established lower limit of quantification (LLOQ). The Biomarker statistical analysis plan (SAP) prospectively defined a criterion for using a biomarker in the analysis that required the biomarker to be above the limit of quantification in at least 80% of the test samples. An expanded panel of 190 biomarkers (Table 1) was used to confirm the results from the initial panel (described in Example 2).

As the LLOQ's for specific analytes can vary across batches of samples analyzed on the RBM platform at different times, the raw data was normalized across all batches by taking the MIN value for each analyte in each batch, then taking the MAX of the MINs for a new ½ LLOQ. This ½ LLOQ value for each analytes was then used to re-clean the data. The cleaned data was then normalized by taking the Z score of the log (concentration) for each analyte. These values were used in a hierarchical clustering algorithm (OmniViz and NCSS software platform) to identify analytes that were significantly associated with SSc (as compared to normals) based on the following criteria: min fold change of 2 and FDR <0.05. The same statistical procedure was used to identify analytes that associated with diffuse SSc (as compared to limited SSc) and analytes that associated with diffuse subset 1 (D1) vs diffuse subset 2 (D2).

A clustered correlation (heatmap) was used as an overall assessment of data quality. No sample outliers were seen in that analysis. The average pairwise correlation from the sample correlation matrix was also assessed and all samples showed at least an average of 89% correlation to other samples, indicating the biomarker data was consistent across subject samples.

### Results

A fold change cutoff of >2 and p value cutoff of <0.05 was used to identify significant analytes from the full panel of 92 analytes. Table 2 shows the serum analytes where the concentrations were associated with SSc subjects as compared to that in healthy normal subjects. Analytes shown on the left are significantly elevated in SSc as compared to normals (>2-fold change FDR, p<0.05). The fold change (ratio of SSc:Normal) as well as the respective p value (Mann-Whitney FDR with multiple testing correction) is shown on the right.

TABLE 2

| Analyte | Ratio SSc:Normal | MW FDR |
|---|---|---|
| CXCL5 | 7.6 | 0.0007 |
| CCL5 | 5.4 | <10⁻⁹ |
| CCL11 | 5.0 | <10⁻⁹ |
| MPO | 4.8 | 0.00002 |
| BDNF | 4.6 | <10⁻⁹ |
| CCL2 | 4.5 | <10⁻⁹ |
| EGF | 4.0 | <10⁻⁹ |
| IL-17 | 2.0 | <10⁻⁹ |

Table 3 shows serum analytes that were associated with diffuse SSc subjects as compared to limited subjects. Analytes shown on the left are significantly different when comparing diffuse to limited SSc subjects (FDR, p<0.05). Although the fold change for some of these analytes was <2, they contributed to the separation seen via hierarchical cluster analysis. The fold change (ratio of diffuse:limited) as well as the respective p value (Mann-Whitney FDR with multiple testing correction) is given on the right. A p value cutoff of <0.05 was used to identify significant analytes from the full panel of 92 analytes.

TABLE 3

| Analyte | Ratio Diffuse:Limited | MW FDR | SEQ ID NO |
|---|---|---|---|
| IL-17 | 0.55 | 0.0007 | 51 |
| IL-13 | 1.8 | <10⁻⁹ | 21 |
| IgE | 1.9 | <10⁻⁹ | 75 |
| GST | 0.75 | 0.00002 | 83 |

Table 4 shows serum analytes that distinguish the diffuse SSc patient subset (D1) from the rest of the diffuse and limited subjects (D2+L). Analytes shown on the left are significantly different when comparing subset D1 to the rest of the diffuse and limited subjects (D2+L, FDR, p<0.05). Although the fold change for some of these analytes was <2, they contributed to the separation seen via hierarchical cluster analysis.

TABLE 4

| Analyte | Ratio (D1:D2 + L) | MW FDR |
|---|---|---|
| CCL5 | 4.3 | 0.0007 |
| CCL11 | 4.2 | <10⁻⁹ |
| BDNF | 4.0 | <10−9 |
| CXCL5 | 2.9 | 0.00002 |
| CCL2 | 2.3 | <10−9 |
| MPO | 2.2 | <10−9 |
| Fibrinogen | 2.1 | <10−9 |
| VEGF | 2.1 | <10−9 |
| IL-17 | 1.8 | .00009 |
| EGF | 1.8 | <10−9 |
| IL-13 | 1.9 | <10−9 |

The marker set of Table 3 (SEQ ID NOS:21, 51, 75, and 83) was used to distinguish limited vs. diffuse SSc among the 74 SSc patients where IL-13 and IgE are higher in the diffuse SSc patient subset than in the limited SSc patient subset and IL-17 and GST are lower in the diffuse SSc patient subset than in the limited SSc patient subset.

A subset of diffuse SSc patients (17 out of 38 subjects, denoted D1) were identified which clustered separately from the rest of the diffuse SSc and limited SSc subjects (58 subjects, denoted D2+L). D1 subjects were identified by the marker set of Table 4. This marker set could be used to correctly identify a D1 subject with a sensitivity of 95% (16/17) and a specificity of 72% (42/58).

### Example 2

### Sample Collection and Analysis

In order to confirm and further define the markers useful in distinguishing SSc patient subsets, serum from an additional cohort of SSc serum samples were analyzed (University of Michigan). The SSc serum cohort consisted of data from 10 subjects with early progressive (EP) diffuse SSc and 10 subjects with late improving (LI) diffuse SSc. The available clinical parameters included age of onset, peak skin score, lung involvement, peripheral white blood cell count. The serum values for all analytes were compared to data pooled from 20 healthy normal subjects (Centocor internal data).

The sera were analyzed for biomarkers using commercially available assays employing either a 190 analyte (shown in Table 1) multiplex analysis performed by Rules Based Medicine (Austin, Tex.) or single analyte ELISA. All samples were stored at −80° C. until tested. The samples were thawed at room temperature, vortexed, spun at 13,000×g for 5 minutes for clarification and 150 uL was removed for antigen analysis into a master microtiter plate. Analysis was performed in a Luminex 100 instrument and the resulting data stream was interpreted using data analysis software from NCSS. For each multiplex, both calibrators and controls were run.

Testing results were determined first for the high, medium and low controls for each multiplex to ensure proper assay performance. Unknown values for each of the analytes localized in a specific multiplex were determined using 4 and 5 parameter, weighted and non-weighted curve fitting algorithms included in the data analysis package.

Each of the 190 biomarkers has an established lower limit of quantification (LLOQ). The Biomarker statistical analysis plan (SAP) prospectively defined a criterion for using a biomarker in the analysis that required the biomarker to be above the limit of quantification in at least 80% of the test samples.

As the LLOQ's for specific analytes can vary across batches of samples analyzed on the RBM platform at different times, the raw data was normalized across all batches by taking the MIN value for each analyte in each batch, then taking the MAX of the MINs for a new ½ LLOQ. This ½ LLOQ value for each analytes was then used to re-clean the data. The cleaned data was then normalized by taking the Z score of the log (concentration) for each analyte. These values were used in a hierarchical clustering algorithm (OmniViz and NCSS software platform) to identify analytes that were significantly associated with SSc (as compared to normals) based on the following criteria: min fold change of 2 and FDR <0.05. The same statistical procedure was used to identify analytes that associated with EP SSc (as compared to LI SSc).

A clustered correlation (heatmap) was used as an overall assessment of data quality. No sample outliers were seen in that analysis. The average pairwise correlation from the sample correlation matrix was also assessed and all samples showed at least an average of 89% correlation to other samples, indicating the biomarker data was consistent across subject samples.

### Results

A fold change cutoff of >2 and p value cutoff of <0.05 was used to identify significant analytes from the full panel of 190 analytes. Table 6 shows the serum analytes where the concentrations were associated with SSc subjects as compared to that in healthy normal subjects. Analytes shown on the left are significantly elevated in SSc as compared to normals (>2-fold change FDR, p<0.05). The fold change (ratio of SSc:Normal) as well as the respective p value (Mann-Whitney FDR with multiple testing correction) is shown on the right.

TABLE 5

| | Ratio | | SEQ ID | |
|---|---|---|---|---|
| Analyte | (SSc:Normal) | MW FDR | NO: | Full Name |
| CD40 Ligand | 330.95 | 0.00001 | 1 | |
| Thrombospondin 1 | 197.52 | 0.00001 | 2 | |
| EGF | 170.23 | 0.00008 | 3 | |
| CgA | 123.73 | 0.00002 | 4 | |
| PDGF BB | 82.14 | 0.00001 | 5 | platelet derived growth factor BB |
| CCL5 | 54.97 | 0.00001 | 6 | |
| BDNF | 37.07 | 0.00001 | 7 | |
| TGF-a | 28.84 | 0.00001 | 8 | Transforming growth factor alpha |
| Epiregulin | 25.39 | 0.00001 | 9 | |
| HB EGF | 20.29 | 0.00001 | 10 | Heparin binding EGF like growth |
| | | | | factor |
| Amphiregulin | 19.40 | 0.00000 | 11 | |
| CRP | 17.82 | 0.00026 | 12 | |
| CCL13 | 15.04 | 0.00001 | 13 | MCP-4 |
| TECK | 13.98 | 0.00001 | 14 | Thymus expressed chemokine |
| CXCL5 | 10.74 | 0.00001 | 15 | |
| NT proBNP | 10.32 | 0.00001 | 16 | N-terminal pro Brain Naturetic |
| | | | | peptide |
| IL 1ra | 8.84 | 0.00001 | 17 | |
| PLGF | 8.72 | 0.00001 | 18 | placental growth factor |
| MMP 1 | 8.30 | 0.00002 | 19 | matrix metalloprotease 1 |
| PAI 1 | 7.84 | 0.00001 | 20 | |
| IL 13 | 6.75 | 0.00395 | 21 | |
| IL 3 | 6.26 | 0.00537 | 22 | |
| CCL8 | 5.63 | 0.00001 | 23 | MCP-2 |
| Apolipoprotein E | 5.49 | 0.00046 | 24 | |
| CCL11 | 5.20 | 0.00002 | 25 | |
| CCL2 | 4.77 | 0.00003 | 26 | |
| Osteopontin | 4.75 | 0.00002 | 27 | |
| MMP9 Total | 4.44 | 0.00001 | 28 | Matrix metalloprotease 9 total |
| GRO alpha | 4.44 | 0.00020 | 29 | |
| S100A12 | 4.43 | 0.00035 | 30 | ENRAGE |
| PAR | 4.36 | 0.00241 | 31 | Pulmonary and Activation |
| | | | | Regulated |
| HGF | 4.22 | 0.00001 | 32 | Hepatocyte Growth Factor |
| VEGF | 4.00 | 0.00001 | 33 | |
| SOD | 3.78 | 0.00100 | 34 | |
| MIP1F1 | 3.75 | 0.00001 | 35 | Myeloid Progenitor Inhibitory |
| | | | | Factor |
| IL 12p40 | 3.74 | 0.00092 | 36 | |
| S100b | 3.60 | 0.01948 | 37 | |
| Ferritin | 3.56 | 0.05464 | 38 | |
| Growth Hormone | 3.45 | 0.04331 | 39 | |
| CKMB | 3.39 | 0.01972 | 40 | Creatine Kinase MB |
| HSP | 3.28 | 0.01627 | 41 | Heat Shock Protein 60 |
| MIP 1beta | 3.28 | 0.00046 | 42 | |
| TIMP 1 | 3.24 | 0.00001 | 43 | Tissue Inhibitor of |
| | | | | Metalloproteases |
| MPO | 3.11 | 0.00017 | 44 | |
| C peptide | 3.08 | 0.00037 | 45 | |
| TNF alpha | 3.03 | 0.05987 | 46 | Tumor necrosis Factor alpha |
| CXCL9 | 2.98 | 0.00011 | 47 | Gamma Interferon induced |
| | | | | Monokine |
| FAS | 2.96 | 0.00001 | 48 | |
| Haptoglobin | 2.90 | 0.00055 | 49 | |
| TRAIL R3 | 2.30 | 0.00017 | 50 | |
| IL-17 | 2.86 | 0.00001 | 51 | |
| MMPI | 2.27 | 0.00100 | 52 | Matrix metalloprotease 7 |
| IL10 | 2.27 | 0.02262 | 53 | |
| IL 16 | 2.23 | 0.00004 | 54 | |
| BLC | 2.21 | 0.00090 | 55 | |
| Insulin | 2.20 | 0.02674 | 56 | B Lymphocyte Chemoattractant |
| von Willebrand | 2.17 | 0.00010 | 57 | |
| Factor | | | | |
| CD40 | 2.14 | 0.00007 | 58 | |
| Stem Cell Factor | 2.11 | 0.00071 | 59 | |
| IL 7 | 2.11 | 0.00963 | 60 | |
| Apolipoprotein H | 2.10 | 0.00001 | 61 | |
| HCC 4 | 2.02 | 0.03342 | 62 | |
| CCL3 | 1.98 | 0.00126 | 63 | MIP 1alpha |
| sRAGE | 1.96 | 0.01948 | 64 | |
| Thrombomodulin | 1.96 | 0.00007 | 65 | |
| IL 8 | −2.61 | 0.02920 | 66 | |
| Apolipoprotein A2 | −3.44 | 0.00001 | 67 | |
| Alpha 2 | −3.69 | 0.05152 | 68 | |
| Macroglobulin | | | | |
| CTGF | −4.57 | 0.00000 | 69 | Connective_Tissue_Growth_Factor |
| ACTH | −6.97 | 0.00000 | 70 | Adrenocorticotropic Hormone |
| SGOT | −7.74 | 0.00001 | 71 | |
| IL 11 | −8.21 | 0.00001 | 72 | |
| IGF 1 | −12.53 | 0.00007 | 73 | |
| NrCAM | −13.56 | 0.00044 | 74 | |
| IgE | −29.35 | 0.00006 | 75 | |
| Angiotensinogen | −177.97 | 0.00001 | 76 | |

Table 6 shows serum analytes that were associated with EP diffuse SSc subjects as compared to LI diffuse subjects. Analytes shown on the left are significantly different when comparing diffuse to limited SSc subjects (FDR, p<0.05). The fold change (ratio of EP:LI) as well as the respective p value (Mann-Whitney FDR with multiple testing correction) is given on the right. A p value cutoff of <0.05 was used to identify significant analytes from the full panel of 190 analytes.

TABLE 6

| | | | SEQ ID |
|---|---|---|---|
| Analyte | Ratio (EP:LI) | MW FDR | NO: |
| FABP | 12.72 | 0.015278 | 77 |
| CRP | 8.03 | 0.002174 | 12 |
| CKMB | 4.83 | 0.012574 | 40 |
| IL 6 | 4.36 | 0.031801 | 78 |
| Myoglobin | 3.97 | 0.018543 | 79 |
| Ferritin | 2.68 | 0.040888 | 38 |
| ANG2 | 2.51 | 0.006672 | 80 |
| SGOT | 2.49 | 0.012869 | 71 |
| MMP 3 | 1.83 | 0.036795 | 81 |
| Haptoglobin | 1.64 | 0.036795 | 49 |
| TIMP 1 | 1.58 | 0.011332 | 43 |
| TTR | −1.63 | 0.014548 | 82 |
| HSP60 | −4.26 | 0.037384 | 41 |
| IGF 1 | −11.76 | 0.003779 | 73 |

The marker set shown in Table 5 was used to distinguish patients diagnosed with SSc from normals with a sensitivity of 100% (20/20 SSc identified) and a specificity of 100% (20/20 HV identified). A determination is made as to which of the markers shown in Table 6 correlate with subject clinical parameters (i.e., skin score, lung function, years since disease onset, etc.) to generate a marker set that is specific to SSc disease progression.

The marker set shown in Table 6 was used to distinguish patients diagnosed with EP SSc from LI SSc with a sensitivity of 90% (9/10 EP identified) and a specificity of 90% (9/10 HV identified).

The subjects were also clustered based on the marker set identified previously from the first serum cohort that distinguished the two subsets of diffuse patients (D1 vs D2+L). The subjects in this second cohort were stratified using the following marker set from Table 2: CXCL5/ENA-78, CCL2/MCP-1, CCL5/RANTES, CCL11/Eotaxin, brain-derived neurotrophic factor (BDNF), myeloperoxidase, IL-17, and epidermal growth factor (EGF). In doing so, two diffuse patient subsets were identified that corresponded to subjects high and low for all of the above markers. The two patient subsets were not differentiated by EP and LI status (each subset contained both EP and LI subjects).

The establishment of disease related serum biomarkers clinically relevant to SSc would enable optimized patient randomization for clinical trials. While the markers identified in the initial multiplex assessment were confirmed in this second cohort, by using a high sensitivity extended multi-analyte panel, an additional panel of markers that differentiates the SSc population from healthy normals was further identified. In addition, a marker set was identified that defines EP SSc subjects from LI subjects. Confirmation of this EP v. LI marker set in an independent cohort is warranted; however, this initial multiplex assessment of serum proteins allows for both early diagnosis of SSc as well as stratification of diffuse SSc patients. While the existence of two clinically distinct subsets of SSc (EP and LI) has been previously described, the present invention describes evidence that these subsets are also serologically different. The existence of two serologically distinct subsets of diffuse SSc should be considered in the frame of randomized clinical trials pending further investigation into its correlation with SSc clinical course, outcome and mortality. In addition to the potential for clinical application, this strategy will also provide novel insight into the modulation of disease specific immune markers during disease evolution and during the treatment phase of clinical studies.

It will be clear that the invention can be practiced otherwise than as particularly described in the foregoing description and examples. Numerous modifications and variations of the present invention are possible in light of the above teachings and, therefore, are within the scope of the appended claims.

1. A method for determining whether a subject is suffering from SSc, the method comprising:
a) obtaining a sample from the subject;
b) determining a concentration of at least one serum marker selected from the group consisting of all or a portion of the amino acid sequences of SEQ ID NOS:1-62 and 66-76; and
c) comparing said concentration with a standard or reference concentration derived from normal control subjects, wherein if the concentration of the marker is about two-fold or more different than the reference concentration, the subject is identified as having SSc.

2. The method of claim 1, wherein the concentration of all or a portion of SEQ ID NOS: 1-16 and 73-76 are determined and compared with the standard or reference concentrations for all or a portion of SEQ ID NOS: 1-16 and 73-76.

3. The method of claim 1, wherein the subject having been classified as having SSc, is further subclassified as having limited SSc or diffuse SSc, the method further comprising:
a) determining the concentrations of all or a portion of the amino acid sequences of SEQ ID NOS: 21, 51, 75 and 83 in the sample obtained from the subject; and
b) comparing the concentrations of all or a portion of the amino acid sequences of SEQ ID NOS: 21, 51, 75 and 83 in the subject sample from the patient diagnosed with SSc to a standard representing patients diagnosed with limited SSc; wherein if the concentrations of all or a portion of SEQ ID NOS: 51 and 83 are lower than in the standard representing patients diagnosed with limited SSc, and the concentrations of all or a portion of SEQ ID NOS: 21 and 75 are higher than in the standard representing patients diagnosed with limited SSc, the patient is classified as having diffuse SSc.

4. A method for determining whether a subject suffering from or diagnosed with SSc is further subclassified as having limited SSc or diffuse SSc, the method comprising:
a) obtaining a sample from the subject;
b) determining the concentration of all or a portion of one or more of the amino acid sequences of SEQ ID NOS: 21, 51, 75 and 83 in the sample obtained from the subject; and
c) comparing the concentrations of one or more of all or a portion of the amino acid sequences of SEQ ID NOS: 21, 51, 75 and 83 in the subject sample from the patient diagnosed with SSc to a standard representing patients diagnosed with limited SSc; wherein if the concentrations of all or a portion of SEQ ID NOS: 51 and/or 83 are lower than in the standard representing patients diagnosed with limited SSc, and/or the concentrations of all or a portion of SEQ ID NOS: 21 and/or 75 are higher than in the standard representing patients diagnosed with limited SSc, the patient is classified as having diffuse SSc.

5. The method of claim 4, further comprising determining whether a subject suffering from diffuse SSc can be further subclassified as EP diffuse SSC or LI diffuse SSC, comprising:
a) determining a concentration of all or a portion of the amino acid sequences of one or more of SEQ ID NOS: 12, 38, 40, 41, 71, 73, and 77-80 in the sample; and
b) comparing said concentrations with reference or standard concentrations derived from patients diagnosed with diffuse SSc; wherein if the concentrations of all or a portion of SEQ ID NOS: 41 and/or 73 are lower than in the standard representing patients diagnosed with diffuse SSc, the subject is classified as having EP diffuse SSc, and if the concentrations of all or a portion of one or more of SEQ ID NOS: 12, 38, 40, 71, and 77-80 are higher than in the standard representing patients diagnosed with diffuse SSc, the subject is classified as having EP diffuse SSc.

6. The method of claim 5, wherein in the comparing step, if the concentration of the marker is about two-fold or more different than the reference concentration, the subject is classified as having EP diffuse SSc.

7. A method for determining whether a subject suffering from diffuse SSc can be further subclassified as EP diffuse SSC or LI diffuse SSC, the method comprising:
a) obtaining a sample from the subject;
b) determining a concentration of all or a portion of the amino acid sequences of one or more of SEQ ID NOS: 12, 38, 40, 41, 71, 73, and 77-80 in the sample; and
c) comparing said concentrations with reference or standard concentrations derived from patients diagnosed with diffuse SSc; wherein if the concentrations of all or a portion of SEQ ID NOS: 41 and/or 73 are lower than in the standard representing patients diagnosed with diffuse SSc, the subject is classified as having EP diffuse SSc, and if the concentrations of all or a portion of one or more of SEQ ID NOS: 12, 38, 40, 71, and 77-80 are higher than in the standard representing patients diagnosed with diffuse SSc, the subject is classified as having EP diffuse SSc.

8. The method of claim 7, wherein in the comparing step, if the concentration of the marker is about two-fold or more different than the reference concentration, the subject is classified as having EP diffuse SSc.

9. A method of monitoring the therapeutic response of a patient previously classified as having diffuse or limited SSc, comprising
a) obtaining a fluid sample from the patient;
b) determining a concentration of at least one serum marker selected from the group consisting all or a portion of the amino acid sequences of SEQ ID NOS: 1-62 and 66-76; and
c) comparing said concentration with a standard or reference concentration derived from normal control subjects, wherein if the concentration of the marker is less than about two-fold different than the reference concentration, the subject is identified as having a therapeutic response to SSc.

10. The method of claim 9, further comprising, prior to the obtaining step, treating the patient with a potential therapy for SSc.

11. A computer-based system for diagnosing a SSc in a subject, comprising means for comparing values from a dataset of a patient to a diagnostic index or an algorithm, wherein the dataset comprises concentrations of one or more markers selected from the group consisting of all or a portion of the amino acid sequences of SEQ ID NOS: 1-62 and 66-76.

12. The computer based system of claim 11, wherein the computer-based system is a trained neural network for processing a patient dataset and produces an output wherein the dataset includes one or more concentrations selected from the group consisting of all or a portion of SEQ ID NOS: 1-62 and 66-76.

13. A computer-based system for further subclassifying a subject diagnosed with diffuse SSc as EP or LI, comprising means for comparing values from a dataset of a patient to a diagnostic index or an algorithm, wherein the dataset comprises concentrations of one or more markers selected from the group consisting of all or a portion of the amino acid sequences of SEQ ID NOS: 12, 38, 40, 41, 71, 73, and 77-80.

14. The computer based system of claim 13, wherein the computer-based system is a trained neural network for processing a patient dataset and produces an output wherein the dataset includes one or more concentrations selected from the group consisting of all or a portion of SEQ ID NOS: 12, 38, 40, 41, 71, 73, and 77-80.

15. A diagnostic device capable of detecting serum markers in a sample obtained from a subject suspected of having SSc, comprising a means for detecting marker concentrations selected from the group consisting of all or a portion of the amino acid sequences of SEQ ID NOS: 1-62 and 66-76.

16. The device of claim 15, wherein the device compares the information produced by detection of all or a portion of at least one of the amino acid sequences of SEQ ID NOS: 1-62 and 66-76 into an algorithm for diagnosing and classifying a subject with SSc.

17. A diagnostic device capable of processing and/or detecting serum markers in a sample obtained from a subject determined as having SSc according to the method of claim 1, comprising a means for detecting marker concentrations of all or a portion of the amino acid sequences of SEQ ID NOS: 12, 38, 40, 41, 71, 73, and 77-80.

18. The device of claim 17, wherein the device compares the information produced by detection of all or a portion of at least one of the amino acid sequences of SEQ ID NOS: 12, 38, 40, 41, 71, 73, and 77-80 into an algorithm for subclassifying a subject with diffuse SSc as EP or LI.

19. A kit comprising the device of claim 17 capable of processing and/or detecting markers in a sample obtained from a subject, wherein the marker concentration is selected from the group consisting of all or a portion of the amino acid sequences of SEQ ID NOS: 1-62 and 66-76.

20. The kit of claim 19, wherein the processed and/or detected markers are used to calculate an index number or in an algorithm for diagnosing and subclassifying a subject suspected of having SSc.

21. A kit comprising the device of claim 17 capable of processing and/or detecting markers in a sample obtained from a subject, wherein the marker concentration is selected from the group consisting of all or a portion of the amino acid sequences of SEQ ID NOS: 12, 38, 40, 41, 71, 73, and 77-80.

22. The kit of claim 21, wherein the processed and/or detected markers are used to calculate an index number or in an algorithm for diagnosing and subclassifying a subject with diffuse SSc as EP or LI.

23. A method for treating a subject identified as suffering from SSc by the method of claim 1 with a potential therapy, the method comprising
treating the subject identified as having SSc with the potential therapy.

24. (canceled)
